(19) United States
(12) Patent Application Publication
(10) Pub. No.: US 2024/0316000 A1
(43) Pub. Date: Sep. 26, 2024

(54) METHODS OF TREATMENT

(71) Applicant: DIFFUSION PHARMACEUTICALS LLC, Charlottesville, VA (US)

(72) Inventors: Robert J. COBUZZI, Chadds Ford, PA (US); Christopher D. GALLOWAY, Castle Rock, CO (US)

(21) Appl. No.: 18/575,201

(22) PCT Filed: Jun. 29, 2022
(86) PCT No.: PCT/US2022/073271

§ 371 Date: Dec. 28, 2023

Related U.S. Application Data
(60) Provisional application No. 63/366,843, filed on Jun. 22, 2022.
(60) Provisional application No. 63/366,840, filed on Jun. 22, 2022.
(60) Provisional application No. 63/364,952, filed on May 18, 2022.
(60) Provisional application No. 63/266,672, filed on Jan. 11, 2022.
(60) Provisional application No. 63/261,826, filed on Sep. 29, 2021.
(60) Provisional application No. 63/216,470, filed on Jun. 29, 2021.

(51) Int. Cl. A61K 31/202; A61P 43/00

(52) U.S. Cl. CPC A61K 31/202; A61P 43/00

(57) ABSTRACT

Provided are novel methods of treatment of conditions such as peripheral tissue oxygenation, interstitial lung disease, dyspnea, ischemia from surgery, brain and pancreatic cancer with increased oxygen diffusivity comprising administering a diffusion enhancing compound, for example, trans sodium crocetinate.

This application claims priority to U.S. Provisional Application No. 63/216,470 filed Jun. 29, 2021, U.S. Provisional Application No. 63/261,826 filed Sep. 29, 2021, U.S. Provisional Application No. 63/266,672 filed Jan. 11, 2022, U.S. Provisional Application No. 63/364,952 filed May 18, 2022, U.S. Provisional Application No. 63/366,840 filed Jun. 22, 2022, and U.S. Provisional Application No. 63/366,843 filed Jun. 22, 2022, each of which is hereby incorporated by reference in its entirety.

### FIELD

Provided are novel methods of treatment comprising administering a diffusion enhancing compound, for example, trans sodium crocetinate.

### BACKGROUND

Carotenoids are a class of hydrocarbons consisting of isoprenoid units. The backbone of the molecule consists of conjugated carbon-carbon double and single bonds, and can have pendant groups. Carotenoids such as crocetin and trans sodium crocetinate (TSC) are known to increase the diffusivity of oxygen in water.

U.S. Pat. No. 6,060,511 relates to trans sodium crocetinate (TSC) and its uses. The patent covers various uses of TSC such as improving oxygen diffusivity and treatment of hemorrhagic shock. U.S. Pat. No. 7,759,506 relates to synthesis methods for making bipolar trans carotenoids (BTC), including bipolar trans carotenoid salts (BTCS), and methods of using them. U.S. Pat. No. 8,030,350 relates to improved BTC synthesis methods and novel uses of the BTC. U.S. Pat. No. 8,293,804 relates to the use of bipolar trans carotenoids as a pretreatment and in the treatment of peripheral vascular disease. U.S. Pat. No. 8,206,751 relates to a new class of therapeutics that enhance small molecule diffusion. U.S. application Ser. No. 12/801,726 relates to diffusion enhancing compounds and their use alone or with thrombolytics.

New methods for improving peripheral tissue oxygenation and treating a disease or condition characterized by hypoxia are needed.

### SUMMARY

Provided is a method of treating a disease or condition in a patient (e.g., a human) in need thereof, wherein the method comprises administering a therapeutically effective amount of a diffusion enhancing compound to the patient.

Further provided is a method of improving (e.g., increasing) peripheral tissue oxygenation in a patient in need thereof (e.g., a human, e.g., a human with interstitial lung disease), wherein the method comprises administering a therapeutically effective amount of a diffusion enhancing compound to the patient. For instance, provided is a method of improving (e.g., increasing) peripheral tissue oxygenation without causing hyperoxygenation in a patient in need thereof (e.g., a human, e.g., a human with a disease or condition characterized by hypoxia, e.g., a human with interstitial lung disease), wherein the method comprises administering a therapeutically effective amount of a diffusion enhancing compound to the patient.

Further provided is a method of treating a disease or condition characterized by hypoxia in a patient in need thereof (e.g., a human, e.g., a human with interstitial lung disease), wherein the method comprises administering a therapeutically effective amount of a diffusion enhancing compound to the patient.

Further provided is a method of treating interstitial lung disease in a patient (e.g., a human) in need thereof, wherein the method comprises administering a therapeutically effective amount of a diffusion enhancing compound to the patient.

Further provided is a method of increasing distance covered in a 6-minute walk test in a human in need thereof (e.g., a human with interstitial lung disease), wherein the method comprises administering a therapeutically effective amount of a diffusion enhancing compound to the patient.

Further provided is a method of treating dyspnea in a patient in need thereof (e.g., a human with interstitial lung disease), wherein the method comprises administering a therapeutically effective amount of a diffusion enhancing compound to the patient.

Further provided is a method of improving (e.g., increasing) oxygen transfer from the alveoli of the lungs to hemoglobin within red blood cells in a patient in need thereof (e.g., a human, e.g., a human with interstitial lung disease), wherein the method comprises administering a therapeutically effective amount of a diffusion enhancing compound to the patient.

Further provided is a method of increasing oxygen transfer from the lungs to the blood in a patient in need thereof (e.g., a human, e.g., a human with interstitial lung disease), wherein the method comprises administering a therapeutically effective amount of a diffusion enhancing compound to the patient.

Further provided is a method of increasing oxygen transfer from blood to peripheral tissue in a patient in need thereof (e.g., a human, e.g., a human with interstitial lung disease), wherein the method comprises administering a therapeutically effective amount of a diffusion enhancing compound to the patient.

Further provided is a method of improving heart rate recovery in a patient in need thereof (e.g., a human, e.g., a human with interstitial lung disease), wherein the method comprises administering a therapeutically effective amount of a diffusion enhancing compound to the patient.

Further provided is a method of treating, preventing, or reducing the amount of ischemia resulting from surgery in a patient in need thereof, wherein the method comprises administering before, during, or after surgery a therapeutically effective amount of a diffusion enhancing compound to the patient.

Further provided is a method of treating cancer (including brain and pancreatic cancer) in a patient in need thereof, wherein the method comprises administering a diffusion enhancing compound to the patient with chemotherapy and/or radiotherapy. For instance, provided is a method of treating a solid tumor in a patient in need thereof, wherein the method comprises administering a diffusion enhancing compound to the patient with chemotherapy and/or radiotherapy.

Further provided is a method for reducing effects of anaerobic metabolism (e.g., a method for increasing blood pH, a method for decreasing lactate (e.g., in the blood), and/or a method for decreasing lactic acid (e.g., in the blood)) in a patient in need thereof, wherein the method comprises administering a therapeutically effective amount of a diffusion enhancing compound to the patient. Further provided is a method for improving post-exercise recovery in a patient in need thereof, wherein the method comprises administering a therapeutically effective amount of a diffusion enhancing compound to the patient. Further provided is a method for enhancing performance when respiration/exertion is increased or stressed (e.g., at altitude above sea level, e.g., at high altitude), a method for increasing aerobic metabolism, and/or a method for increasing endurance during physical activity (e.g., running, walking, or lifting) in a patient in need thereof, wherein the method comprises administering a therapeutically effective amount of a diffusion enhancing compound to the patient.

In any of the above methods, the diffusion enhancing compound may be a bipolar trans carotenoid, advantageously a bipolar trans carotenoid salt (e.g., TSC). In a further embodiment, the bipolar trans carotenoid salt is formulated with a cyclodextrin. The diffusion enhancing compound is advantageously administered IV or IM. If the diffusion enhancing compound is TSC, a dose of 0.05-5 mg/kg, e.g., 0.05-2.5 mg/kg, e.g., 2-2.5 mg/kg, or 2.5-5, e.g., 3-5 mg/kg, may be administered.

Further provided is a kit comprising:
a) a container comprising a diffusion enhancing compound such as TSC, and
b) instructions for using the diffusion enhancing compound in a method disclosed herein by administering the diffusion enhancing compound, for instance, by administering the diffusion enhancing compound at a dose of 0.05-5 mg/kg, e.g., 0.05-2.5 mg/kg or 2.5-5 mg/kg, e.g., 3-5 mg/kg, to the patient. The kit may be used for any of the methods described herein.

Further provided is a double chamber container or syringe for separately holding in the two chambers (and combining just before administration): a) a solid, in particular a lyophilizate of a diffusion enhancing compound such as TSC, and b) a liquid reconstitution medium therefor such as water for injection. The container or syringe may be used in any of the methods disclosed herein.

### BRIEF DESCRIPTION OF THE DRAWINGS

FIG. 1 shows median change in TcpO₂ values for trans sodium crocetinate (TSC) dose minus placebo in Period 1.

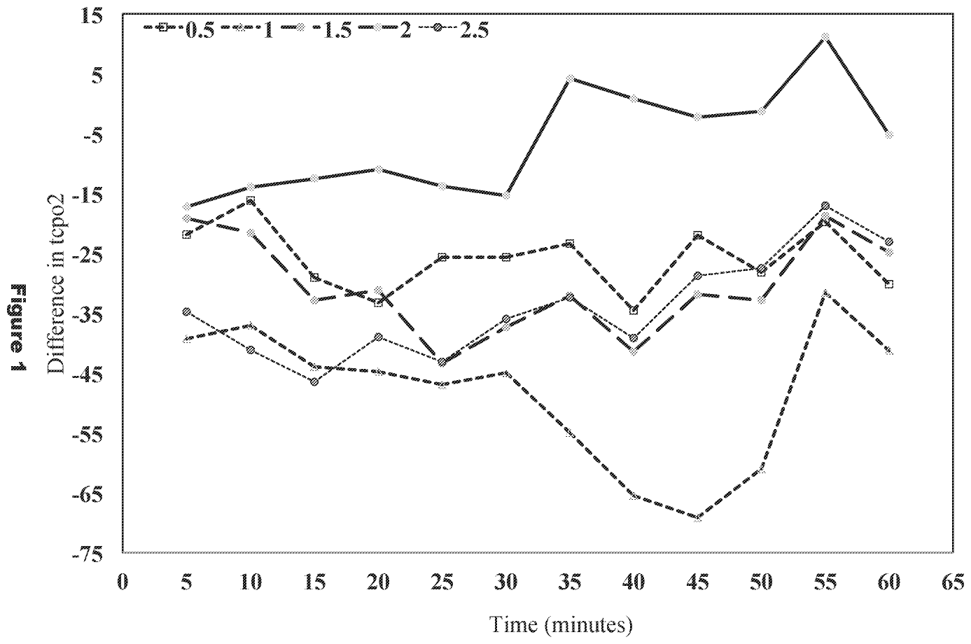

FIG. 2 shows median change in TcpO₂ values for trans sodium crocetinate (TSC) dose minus placebo in Period 2.

FIG. 3 shows trans sodium crocetinate (TSC) concentration profiles for subjects.

FIG. 4 shows simulated trans sodium crocetinate (TSC) concentration profiles for a subject weighing 75 kg.

FIG. 5 shows median TcpO₂ values during Period 2. Axes for each graph are Change in Partial Pressure Oxygen (mm Hg) versus Time (minutes).

### DETAILED DESCRIPTION

As used herein, patient includes human and non-human. Preferably, a patient is human.

FiO₂ is fraction of inspired oxygen. PaO₂ is arterial partial pressure of oxygen. SpO₂/FiO₂ may be used as a non-invasive alternative to PaO₂/FiO₂.

TSC has been shown to have beneficial effects in hypoxemic situations. For example, TSC has been shown to increase whole-body oxygen consumption after hemorrhagic shock in rats. TSC increases oxygen levels in hypoxemic states, both in arterial and tissue levels, but does not in normoxic states. TSC has these effects because it increases the diffusivity of oxygen through plasma. The diffusion rate is known to be affected by both concentration and diffusivity (i.e. Fick's law).

Not only does TSC seem to enhance systemic oxygenation of tissues, but it may also affect passage of oxygen from the alveoli to erythrocytes to enhance the oxygen carrying capacity of blood in acute respiratory distress syndrome (ARDS).

Blood oxygen level is a measure of how much oxygen your red blood cells are carrying. Blood oxygen level can be measured with an arterial blood gas (ABG) test and/or a pulse oximeter. A measurement of blood oxygen level is called oxygen saturation level. The measurement may be referred to as SaO₂. Blood oxygen level may also be called PaO₂ when an ABG test is done or an O₂ sat (SpO₂) when it is measured with a pulse oximeter. A normal ABG level for healthy lungs falls between 80 and 100 millimeters of mercury (mm Hg). If a pulse oximeter is used, a normal reading may be between 95 and 100%. A below-normal blood oxygen level is hypoxemia. As used herein, hypoxemia includes an ABG level below 80 mm Hg (e.g., at or below 70 mm Hg, e.g., at or below 60 mm Hg, e.g., at or below 50 mm Hg, e.g., below 50 mm Hg) and/or an SpO₂ below 95% percent (e.g., below 90%).

The methods described herein include administration of a therapeutically effective amount of a diffusion enhancing compound such as TSC.

Previous work with TSC reported that it exhibited a U-shaped dosage curve. Specifically, Manabe et al., J Neurosurgery, 2010, 113 (4), 802-809, in testing the effect of TSC on cerebral infarct volume in a model of permanent (24-hour) focal ischemia at dosages ranging from 0.023 to 4.580 mg/kg, found maximal protective effect at an intermediate dosage of 0.092 mg/kg. Mohler et al., Vasc Med., 2011, 16 (5), 346-353, in administering TSC ranging from 0.25 mg/kg to 2.0 mg/kg to patients with peripheral artery disease, found a peak increase in peak walking time after 5 days of treatment in the 1.50 mg/kg dosing arm. Mohler reported the greatest increase in claudication onset time was observed at the lowest dose of TSC of 0.25 mg/kg. U.S. Pat. No. 8,030,350 reported that for HCT116 human colon carcinoma tumors on mice, TSC tested at doses 0.07 mg/kg, 0.14 mg/kg, 0.18 mg/kg, 0.28 mg/kg, 0.54 mg/kg, and 1.35 mg/kg with radiotherapy, optimal doses were 0.07 mg/kg, 0.14 mg/kg, and 0.18 mg/kg. U.S. Pat. No. 11,185,523 discusses a “low” dose range of 0.15-0.35 mg/kg and a “high” dose range of 0.75-2.0 mg/kg for humans.

Applicant has now found that administration of trans sodium crocetinate unexpectedly enhances delivery of oxygen to tissues with low oxygen levels in humans more effectively at higher dosages, without causing hyperoxia.

Diffusion enhancing compounds for use in the methods described herein include bipolar trans carotenoid salts having the formula:

YZ-TCRO-ZY,
where:
Y=a cation which can be the same or different,
Z=a polar group which can the same or different and which is associated with the cation,
TCRO=a linear trans carotenoid skeleton with conjugated carbon-carbon double bonds and single bonds, and having pendant groups X, wherein the pendant groups X, which can be the same or different, are a linear or branched hydrocarbon group having 10 or less carbon atoms, or a halogen.

Advantageously, the bipolar trans carotenoid is the all trans form of crocetin (trans-crocetin), which may be in acid or pharmaceutically acceptable salt form. Trans sodium crocetinate (TSC) (e.g., synthetic TSC) is shown as Formula I below.

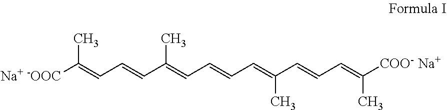

In one embodiment, the absorbency (e.g., in an aqueous solution) of the bipolar trans carotenoid salt (e.g., trans sodium crocetinate) at the highest peak which occurs in the visible wavelength range divided by the absorbency of a peak occurring in the ultraviolet wavelength range is greater than 7 (e.g., 7 to 8.5), e.g., greater than 7.5 (e.g., 7.5 to 9, e.g., 7.5 to 8.5), e.g., greater than 8 (e.g., 8 to 8.8), e.g., greater than 8.5. In another embodiment, the absorbency (e.g., in an aqueous solution) of the TSC at the highest peak which occurs in the visible wavelength range divided by the absorbency of a peak occurring in the ultraviolet wavelength range is greater than 7 (e.g., 7 to 8.5), e.g., greater than 7.5 (e.g., 7.5 to 9, e.g., 7.5 to 8.5), e.g., greater than 8 (e.g., 8 to 8.8), e.g., greater than 8.5.

The bipolar trans carotenoid salt (e.g., trans sodium crocetinate) is at least 90% pure as measured by high performance liquid chromatography (HPLC), e.g., ≥95% pure as measured by HPLC, e.g., ≥96% pure as measured by HPLC. In an advantageous embodiment, the TSC is at least 90% pure as measured by high performance liquid chromatography (HPLC), e.g., ≥95% pure as measured by HPLC, e.g., ≥96% pure as measured by HPLC.

In an advantageous embodiment, the bipolar trans carotenoid salt is in a composition also comprising a cyclodextrin. For instance, wherein TSC is in a composition also comprising a cyclodextrin (e.g., wherein the TSC is in a lyophilized composition with a cyclodextrin).

Advantageously, the cyclodextrin is gamma-cyclodextrin. For instance, the bipolar trans carotenoid salt is TSC which is in a composition also comprising gamma-cyclodextrin (e.g., wherein the TSC is in a lyophilized composition with gamma-cyclodextrin).

In an embodiment of the invention, the composition further comprises mannitol.

The diffusion enhancing compound is administered intravenously or intramuscularly (e.g., as an intravenous injection or infusion or intramuscular injection).

Advantageously, the diffusion enhancing compound is admixed with sterile water for injection to form an injection. TSC is administered intravenously or intramuscularly (e.g., as an intravenous injection or infusion or intramuscular injection). For instance, wherein TSC is admixed with sterile water for injection to form an injection.

Advantageously, the diffusion enhancing compound is TSC and is administered at a dose of 0.05-5 mg/kg, e.g., 0.05-2.5 mg/kg, e.g., 2-2.5 mg/kg, or 2.5-5 mg/kg, e.g., 3-5 mg/kg.

In another embodiment, provided is a diffusion enhancing compound (e.g., a bipolar trans carotenoid salt (e.g., TSC)) for use in any of the methods described herein.

In another embodiment, provided is use of a diffusion enhancing compound (e.g., a bipolar trans carotenoid salt (e.g., TSC)) in the manufacture of a medicament for any of the methods described herein.

In another embodiment, provided is a pharmaceutical composition comprising an effective amount of a diffusion enhancing compound (e.g., a bipolar trans carotenoid salt (e.g., TSC)) for use in any of the methods described herein.

### Compounds and Compositions of the Invention

### Diffusion Enhancing Compounds

Diffusion enhancing compounds include those compounds described in U.S. Pat. Nos. 7,759,506, 8,030,350, 8,901,174 U.S. Pat. Nos. 8,206,751, and 11,185,523, each of which is hereby incorporated by reference in its entirety.

Included are bipolar trans carotenoid compounds having the formula:

YZ-TCRO-ZY

where:
Y=a cation
Z=a polar group which is associated with the cation, and
TCRO-trans carotenoid skeleton,
   such as TSC.

More specifically, the subject invention relates to trans carotenoids including trans carotenoid diesters, dialcohols, diketones and diacids, bipolar trans carotenoids (BTC), and bipolar trans carotenoid salts (BTCS) and uses of such compounds having the structure:

YZ-TCRO-ZY

where:
Y (which can be the same or different at the two ends)=H or a cation other than H, preferably Na⁺ or K⁺ or Li⁺. Y is advantageously a monovalent metal ion. Y can also be an organic cation, e.g., R₄N⁺, R₃S⁺, where R is H, or CₙH₂ₙ₊1 where n is 1-10, advantageously 1-6. For example, R can be methyl, ethyl, propyl or butyl.
Z (which can be the same or different at the two ends)=polar group which is associated with H or the cation. Optionally including the terminal carbon on the carotenoid (or carotenoid related compound), this group can be a carboxyl (COO⁻) group or a CO group (e.g. ester, aldehyde or ketone group), or a hydroxyl group. This group can also be a sulfate group (OSO₃⁻) or a monophosphate group (OPO₃⁻), (OP(OH)O₂⁻), a diphosphate group, triphosphate or combinations thereof. This group can also be an ester group of COOR where the R is CₙH₂ₙ₊₁.
TCRO=trans carotenoid or carotenoid related skeleton (advantageously less than 100 carbons) which is linear, has pendant groups (defined below), and typically comprises “conjugated” or alternating carbon-carbon double and single bonds (in one embodiment, the TCRO is not fully conjugated as in a lycopene). The pendant groups (X) are typically methyl groups but can be other groups as discussed below. In an advantageous embodiment, the units of the skeleton are joined in such a manner that their arrangement is reversed at the center of the molecule. The 4 single bonds that surround a carbon-carbon double bond all lie in the same plane. If the pendant groups are on the same side of the carbon-carbon double bond, the groups are designated as cis (also known as “Z”); if they are on the opposite side of the carbon-carbon bond, they are designated as trans (also known as “E”). Throughout this case, the isomers will be referred to as cis and trans.

The compounds of the subject invention are trans. The cis isomer typically is a detriment—and results in the diffusivity not being increased. In one embodiment, a cis isomer can be utilized where the skeleton remains linear. The placement of the pendant groups can be symmetric relative to the central point of the molecule or can be asymmetric so that the left side of the molecule does not look the same as the right side of the molecule either in terms of the type of pendant group or their spatial relationship with respect to the center carbon.

The pendant groups X (which can be the same or different) are hydrogen (H) atoms, or a linear or branched hydrocarbon group having 10 or less carbons, advantageously 4 or less, (optionally containing a halogen), or a halogen. X could also be an ester group (COO—) or an ethoxy/methoxy group. Examples of X are a methyl group (CH₃), an ethyl group (C₂H₅), a phenyl or single aromatic ring structure with or without pendant groups from the ring, a halogen-containing alkyl group (C1-C10) such as CH₂Cl, or a halogen such as Cl or Br or a methoxy (OCH₃) or ethoxy (OCH₂CH₃). The pendant groups can be the same or different but the pendant groups utilized must maintain the skeleton as linear.

Although many carotenoids exist in nature, carotenoid salts do not. Commonly-owned U.S. Pat. No. 6,060,511 hereby incorporated by reference in its entirety, relates to trans sodium crocetinate (TSC). The TSC was made by reacting naturally occurring saffron with sodium hydroxide followed by extractions that selected primarily for the trans isomer.

The presence of the cis and trans isomers of a carotenoid or carotenoid salt can be determined by looking at the ultraviolet-visible spectrum for the carotenoid sample dissolved in an aqueous solution. Given the spectrum, the value of the absorbence of the highest peak which occurs in the visible wave length range of 380 to 470 nm (the number depending on the solvent used and the chain length of the BTC or BTCS. The addition of pendant groups or differing chain lengths will change this peak absorbance but someone skilled in the art will recognize the existence of an absorbance peak in the visible range corresponding to the conjugated backbone structure of these molecules.) is divided by the absorbency of the peak which occurs in the UV wave length range of 220 to 300 nm can be used to determine the purity level of the trans isomer. When the trans carotenoid diester (TCD) or BTCS is dissolved in water, the highest visible wave length range peak will be at between 380 nm to 470 nm (depending on the exact chemical structure, backbone length and pendant groups) and the UV wave length range peak will be between 220 to 300 nm. According to M. Craw and C. Lambert, Photochemistry and Photobiology, Vol. 38 (2), 241-243 (1983) hereby incorporated by reference in its entirety, the result of the calculation (in that case crocetin was analyzed) was 3.1, which increased to 6.6 after purification.

Performing the Craw and Lambert analysis, using a cuvette designed for UV and visible wavelength ranges, on the trans sodium salt of crocetin of commonly owned U.S. Pat. No. 6,060,511 (TSC made by reacting naturally occurring saffron with sodium hydroxide followed by extractions which selected primarily for the trans isomer), the value obtained averages about 6.8. Performing that test on the synthetic TSC of the subject invention, that ratio is greater than 7.0 (e.g. 7.0 to 8.5), advantageously greater than 7.5 (e.g. 7.5-8.5), most advantageously greater than 8. The synthesized material is a “purer” or highly purified trans isomer.

### Formulation and Administration of the Compounds and Compositions of the Invention

A detailed description of formulation and administration of diffusing enhancing compounds can be found in commonly owned U.S. Pat. No. 8,293,804, U.S. application Ser. No. 12/801,726, and U.S. Pat. No. 8,206,751, each of which is hereby incorporated by reference in its entirety. A detailed description of formulation and administration of diffusing enhancing compounds can also be found in commonly owned U.S. Pat. No. 8,030,350, which is hereby incorporated by reference in its entirety.

A diffusion enhancing compound such as TSC can be administered by various routes for rapid delivery to the hypoxic tissue. For example, the compound, which can be formulated with other compounds including excipients, can be administered at the proper dosage as an intravenous injection (IV) or infusion, or an intramuscular injection (IM).

The IV injection route is an advantageous route for giving TSC for many of the uses of the subject application. Typically, a diffusion enhancing compound such as TSC is administered as soon as possible if a thrombus is believed present.

In addition to intravenous injection, routes of administration for specially formulated trans carotenoid molecules include intramuscular injection, delivery by inhalation, oral administration and transdermal administration.

### Cyclodextrins

In order to administer some pharmaceuticals, it is necessary to add another compound which will aid in increasing the absorption/solubility/concentration of the active pharmaceutical ingredient (API). Such compounds are called excipients, and cyclodextrins are examples of excipients. Cyclodextrins are cyclic carbohydrate chains derived from starch. They differ from one another by the number of glucopyranose units in their structure. The parent cyclodextrins contain six, seven and eight glucopyranose units, and are referred to as alpha, beta and gamma cyclodextrins respectively. Cyclodextrins were first discovered in 1891, and have been used as part of pharmaceutical preparations for several years.

Cyclodextrins are cyclic (alpha-1,4)-linked oligosaccharides of alpha-D-glucopyranose containing a relatively hydrophobic central cavity and hydrophilic outer surface. In the pharmaceutical industry, cyclodextrins have mainly been used as complexing agents to increase the aqueous solubility of poorly water-soluble drugs, and to increase their bioavailability and stability. In addition, cyclodextrins are used to reduce or prevent gastrointestinal or ocular irritation, reduce or eliminate unpleasant smells or tastes, prevent drug-drug or drug-additive interactions, or even to convert oils and liquid drugs into microcrystalline or amorphous powders.

There are a number of cyclodextrins that can be used with the diffusion enhancing compounds disclosed herein. See for example, U.S. Pat. No. 4,727,064, hereby incorporated by reference in its entirety. Advantageous cyclodextrins are γ-cyclodextrin, 2-hydroxylpropyl-γ-cyclodextrin and 2-hydroxylpropyl-β-cyclodextrin, or other cyclodextrins which enhance the solubility of the BTC.

The use of gamma-cyclodextrin with TSC increases the solubility of TSC in water by 3-7 times. Although this is not as large a factor as seen in some other cases for increasing the solubility of an active agent with a cyclodextrin, it is important in allowing for the parenteral administration of TSC in smaller volume dosages to humans (or animals). Dosages of TSC and gamma-cyclodextrin have resulted in aqueous solutions containing as much as 44 milligrams of TSC per ml of solution, with an advantageous range of 20-30 mg/ml of solution. The solutions need not be equal-molar. The incorporation of the gamma cyclodextrin also allows for TSC to be absorbed into the blood stream when injected intramuscularly. Absorption is quick, and efficacious blood levels of TSC are reached quickly (as shown in rats).

The cyclodextrin formulation can be used with other trans carotenoids and carotenoid salts. The subject invention also includes novel compositions of carotenoids which are not salts (e.g. acid forms such as crocetin, crocin or the intermediate compounds noted above) and a cyclodextrin. In other words, trans carotenoids which are not salts can be formulated with a cyclodextrin. Mannitol can be added for osmolality, or the cyclodextrin BTC mixture can be added to isotonic saline (see below).

The amount of the cyclodextrin used is that amount which will contain the trans carotenoid but not so much that it will not release the trans carotenoid. Advantageously, the ratio of cyclodextrin to BTC, e.g., TSC, is 4 to 1 or 5 to 1. See also U.S. Pat. No. 8,974,822, the content of which is hereby incorporated by reference in its entirety.

### Cyclodextrin-Mannitol

A trans carotenoid such as TSC can be formulated with a cyclodextrin as noted above and a non-metabolized sugar such as mannitol (e.g. d-mannitol to adjust the osmotic pressure to be the same as that of blood). Solutions containing over 20 mg TSC/ml of solution can be made this way. This solution can be added to isotonic saline or to other isotonic solutions in order to dilute it and still maintain the proper osmolality.

### Mannitol/Acetic Acid

A BTCS such as TSC can be formulated with mannitol such as d-mannitol, and a mild buffering agent such as acetic acid or citric acid to adjust the pH. The pH of the solution should be around 8 to 8.5. It should be close to being an isotonic solution, and, as such, can be injected directly into the blood stream.

### Water+Saline

A BTCS such as TSC can be dissolved in water (advantageously injectable water). This solution can then be diluted with water, normal saline, Ringer's lactate or phosphate buffer, and the resulting mixture either infused or injected.

### Buffers

A buffer such as glycine, bicarbonate, or sodium carbonate can be added to the formulation at a level of about 50 mM for stability of the BCT such as TSC.

### TSC and Gamma-Cyclodextrin

The ratio of TSC to cyclodextrin is based on TSC:cyclodextrin solubility data. For example, 20 mg/ml TSC, 8% gamma cyclodextrin, 50 mM glycine, 2.33% mannitol with pH 8.2+/−0.5, or 10 mg/ml TSC and 4% cyclodextrin, or 5 mg/ml and 2% cyclodextrin. The ratios of these ingredients can be altered somewhat, as is obvious to one skilled in this art.

Mannitol can be used to adjust osmolality and its concentration varies depending on the concentration of other ingredients. The glycine is held constant. TSC is more stable at higher pHs. pH of around 8.2+/−0.5 is required for stability and physiological compatibility. The use of glycine is compatible with lyophilization. Alternatively, the TSC and cyclodextrin is formulated using a 50 mM bicarbonate buffer in place of the glycine.

### Endotoxin Removal of Gamma-Cyclodextrin

Commercially available pharmaceutical grade cyclodextrin has endotoxin levels that are incompatible with intravenous injection. The endotoxin levels must be reduced in order to use the cyclodextrin in a BTC formulation intended for intravenous injection.

### Kits and Dual Chamber Delivery Systems

The diffusion enhancing compound such as TSC can be lyophilized and put in a vial which can be part of a vial kit system which also includes a vial with diluent such as water for injection, and a syringe for administration.

Dual-chamber delivery systems allow reconstitution of the lyophilized diffusion enhancing compound directly inside the system be it a syringe or a cartridge. The lyophilized diffusion enhancing compound such as TSC is located in one chamber and the diluent (e.g. water for injection) in the other. The drug is reconstituted just before administration. It is a simple and controllable process completed in a few easy steps.

In one embodiment, the diffusion enhancing compound such as TSC is loaded in an auto-injector. An auto-injector (or auto-injector) is a medical device designed to deliver a dose of a particular drug. Most auto-injectors are spring-loaded syringes. By design, auto-injectors are easy to use and are intended for self-administration by patients, or administration by untrained personnel. The site of injection is typically the thigh or the buttocks. The auto-injector typically keeps the needle tip shielded prior to injection and also has a passive safety mechanism to prevent accidental firing (injection). Injection depth can be adjustable or fixed and a function for needle shield removal can be incorporated. Just by pressing a button, the syringe needle is automatically inserted and the drug is delivered.

### Uses of the Compounds and Compositions of the Invention

The diffusion enhancing compound can be administered by various routes. For example, the compound (which can be formulated with other compounds), can be administered at the proper dosage as an intravenous injection or infusion, an intramuscular injection, or in an oral form. The IV injection route is an advantageous route for giving a diffusion enhancing compound such as TSC.

Provided is a method (Method 1) of treating a disease or condition in a patient in need thereof, wherein the method comprises administering a therapeutically effective amount of a diffusion enhancing compound to the patient.

Further provided is a method (Method 2) of improving (e.g., increasing) peripheral tissue oxygenation in a patient in need thereof (e.g., a human, e.g., a human with interstitial lung disease), wherein the method comprises administering a therapeutically effective amount of a diffusion enhancing compound to the patient. For instance, provided is a method of improving (e.g., increasing) peripheral tissue oxygenation without causing hyperoxygenation in a patient in need thereof (e.g., a human, e.g., a human with a disease or condition characterized by hypoxia, e.g., a human with interstitial lung disease), wherein the method comprises administering a therapeutically effective amount of a diffusion enhancing compound to the patient.

Further provided is a method (Method 3) of treating a disease or condition characterized by hypoxia in a patient in need thereof (e.g., a human, e.g., a human with interstitial lung disease), wherein the method comprises administering a therapeutically effective amount of a diffusion enhancing compound to the patient.

Further provided is a method (Method 4) of treating interstitial lung disease in a patient (e.g., a human) in need thereof, wherein the method comprises administering a therapeutically effective amount of a diffusion enhancing compound to the patient.

Further provided is a method (Method 5) of increasing distance covered in a 6-minute walk test in a human in need thereof (e.g., a human with interstitial lung disease), wherein the method comprises administering a therapeutically effective amount of a diffusion enhancing compound to the patient.

Further provided is a method (Method 6) of treating dyspnea in a patient in need thereof (e.g., a human with interstitial lung disease), wherein the method comprises administering a therapeutically effective amount of a diffusion enhancing compound to the patient.

Further provided is a method (Method 7) of improving (e.g., increasing) oxygen transfer from the alveoli of the lungs to hemoglobin within red blood cells in a patient in need thereof (e.g., a human, e.g., a human with interstitial lung disease), wherein the method comprises administering a therapeutically effective amount of a diffusion enhancing compound to the patient.

Further provided is a method (Method 8) of increasing oxygen transfer from the lungs to the blood in a patient in need thereof (e.g., a human, e.g., a human with interstitial lung disease), wherein the method comprises administering a therapeutically effective amount of a diffusion enhancing compound to the patient.

Further provided is a method (Method 9) of increasing oxygen transfer from blood to peripheral tissue in a patient in need thereof (e.g., a human, e.g., a human with interstitial lung disease), wherein the method comprises administering a therapeutically effective amount of a diffusion enhancing compound to the patient.

Further provided is a method (Method 10) of improving heart rate recovery in a patient in need thereof (e.g., a human, e.g., a human with interstitial lung disease), wherein the method comprises administering a therapeutically effective amount of a diffusion enhancing compound to the patient.

Further provided is a method (Method 11) of treating, preventing, or reducing the amount of ischemia resulting from surgery in a patient in need thereof, wherein the method comprises administering to the patient before, during, or after surgery a therapeutically effective amount of a diffusion enhancing compound.

Further provided is a method (Method 12) of treating cancer (including brain and pancreatic cancer) in a patient in need thereof, wherein the method comprises administering a diffusion enhancing compound to the patient with chemotherapy and/or radiotherapy. For instance, provided is a method of treating a solid tumor in a patient in need thereof, wherein the method comprises administering a diffusion enhancing compound to the patient with chemotherapy and/or radiotherapy.

Further provided is a method (Method 13a) for reducing effects of anaerobic metabolism (e.g., a method for increasing blood pH, a method for decreasing lactate (e.g., in the blood), and/or a method for decreasing lactic acid (e.g., in the blood)) in a patient in need thereof, wherein the method comprises administering a therapeutically effective amount of a diffusion enhancing compound to the patient. Further provided is a method (Method 13b) for improving post-exercise recovery in a patient in need thereof, wherein the method comprises administering a therapeutically effective amount of a diffusion enhancing compound to the patient. Further provided is a method (Method 13c) for enhancing performance when respiration/exertion is increased or stressed (e.g., at altitude above sea level, e.g., at high altitude), a method for increasing aerobic metabolism, and/or a method for increasing endurance during physical activity (e.g., running, walking, or lifting) in a patient in need thereof, wherein the method comprises administering a therapeutically effective amount of a diffusion enhancing compound to the patient. Any reference to Method 13 herein is a reference to any one of Method 13a, Method 13b, or Method 13c.

Further provided are any one of Methods 1-13 as follows:
1.1. Any one of Methods 1-13, wherein said diffusion enhancing compound is a bipolar trans carotenoid salt having the formula:

YZ-TCRO-ZY,
where:
Y=a cation which can be the same or different,
Z=a polar group which can the same or different and which is associated with the cation,
TCRO=a linear trans carotenoid skeleton with conjugated carbon-carbon double bonds and single bonds, and having pendant groups X, wherein the pendant groups X, which can be the same or different, are a linear or branched hydrocarbon group having 10 or less carbon atoms, or a halogen.
1.2. Any one of Methods 1-13 or 1.1, wherein the diffusion enhancing compound is trans-crocetin, in acid or pharmaceutically acceptable salt form. For instance, any one of Methods 1-13 or 1.1, wherein the diffusion enhancing compound is trans sodium crocetinate (TSC) (e.g., synthetic TSC).
1.3. Any one of Methods 1-13, 1.1, or 1.2, wherein the absorbency (e.g., in an aqueous solution) of the bipolar trans carotenoid salt (e.g., trans sodium crocetinate) at the highest peak which occurs in the visible wavelength range divided by the absorbency of a peak occurring in the ultraviolet wavelength range is equal to or greater than 7 (e.g., 7 to 8.5), e.g., equal to or greater than 7.5 (e.g., 7.5 to 9, e.g., 7.5 to 8.5), e.g., equal to or greater than 8 (e.g., 8 to 8.8), e.g., greater than 8.5. For instance, wherein the absorbency (e.g., in aqueous solution) of the highest peak which occurs in the visible wavelength range of 380 to 470 nm divided by the absorbency of the peak which occurs in the UV wavelength range of 220 to 300 nm is equal to or greater than 7 (e.g., 7 to 8.5), e.g., equal to or greater than 7.5 (e.g., 7.5 to 9, e.g., 7.5 to 8.5), e.g., equal to or greater than 8 (e.g., 8 to 8.8), e.g., greater than 8.5.
1.4. Any one of Methods 1-13 or 1.1-1.3, wherein the absorbency (e.g., in an aqueous solution) of the TSC at the highest peak which occurs in the visible wavelength range divided by the absorbency of a peak occurring in the ultraviolet wavelength range is equal to or greater than 7 (e.g., 7 to 8.5), e.g., equal to or greater than 7.5 (e.g., 7.5 to 9, e.g., 7.5 to 8.5), e.g., equal to or greater than 8 (e.g., 8 to 8.8), e.g., greater than 8.5. For instance, wherein the absorbency (e.g., in aqueous solution) of the TSC at the highest peak which occurs in the visible wavelength range of 380 to 470 nm divided by the absorbency of the peak which occurs in the UV wavelength range of 220 to 300 nm is equal to or greater than 7 (e.g., 7 to 8.5), e.g., equal to or greater than 7.5 (e.g., 7.5 to 9, e.g., 7.5 to 8.5), e.g., equal to or greater than 8 (e.g., 8 to 8.8), e.g., greater than 8.5.
1.5. Any one of Methods 1-13 or 1.1-1.4, wherein the bipolar trans carotenoid salt (e.g., trans sodium crocetinate) is at least 90% pure as measured by high performance liquid chromatography (HPLC), e.g., ≥95% pure as measured by HPLC, e.g., ≥96% pure as measured by HPLC.
1.6. Any one of Methods 1-13 or 1.1-1.5, wherein the TSC is at least 90% pure as measured by high performance liquid chromatography (HPLC), e.g., ≥95% pure as measured by HPLC, e.g., ≥96% pure as measured by HPLC.
1.7. Any one of Methods 1-13 or 1.1-1.6, wherein the bipolar trans carotenoid salt is in a composition also comprising a cyclodextrin. For instance, wherein TSC is in a composition also comprising a cyclodextrin (e.g., wherein the TSC is in a lyophilized composition with a cyclodextrin).
1.8. Method 1.7, wherein the cyclodextrin is selected from the group consisting of alpha cyclodextrin, beta cyclodextrin, 2-hydroxypropyl-beta-cyclodextrin, 2-hydroxypropyl-gamma-cyclodextrin, and gamma cyclodextrin.
1.9. Method 1.8, wherein the cyclodextrin is selected from the group consisting of alpha cyclodextrin, 2-hydroxypropyl-beta-cyclodextrin, and gamma cyclodextrin.
1.10. Method 1.9, wherein the cyclodextrin is gamma-cyclodextrin. For instance, wherein the bipolar trans carotenoid salt is TSC which is in a composition also comprising gamma-cyclodextrin (e.g., wherein the TSC is in a lyophilized composition with gamma-cyclodextrin).
1.11. Any one of Methods 1-13 or 1.1-1.10, wherein the composition further comprises mannitol.
1.12. Any one of Methods 1-13 or 1.1-1.11, wherein the diffusion enhancing compound is administered intravenously or intramuscularly (e.g., as an intravenous bolus injection or intravenous infusion or intramuscular injection). For instance, any one of Methods 1-13 or 1.1-1.11, wherein the diffusion enhancing compound is admixed with sterile water for injection to form an injection. Any one of Methods 1-13 or 1.1-1.11, wherein TSC is administered intravenously or intramuscularly (e.g., as an intravenous bolus injection or intravenous infusion or intramuscular injection). For instance, any one of Methods 1-13 or 1.1-1.11, wherein TSC is admixed with sterile water for injection to form an injection.
1.13. Any one of Methods 1-13 or 1.1-1.12, wherein the diffusion enhancing compound is administered intravenously (e.g., as an intravenous bolus injection).
1.14. Any one of Methods 1-13 or 1.1-1.13, wherein the diffusion enhancing compound is administered at a dose of 0.05-5 mg/kg, e.g., 0.05-2.5 mg/kg, e.g., 0.1-2 mg/kg, or 2.5-5 mg/kg, e.g., 3-5 mg/kg. For instance, any one of Methods 1-13 or 1.1-1.13, wherein the diffusion enhancing compound is TSC and is administered at a dose of 0.05-5 mg/kg, e.g., 0.05-2.5 mg/kg (e.g., 1-2.5 mg/kg or 1.5-2.5 mg/kg or 0.5 mg/kg or 1 mg/kg or 1.5 mg/kg or 2 mg/kg or 2.5 mg/kg), e.g., 2-2.5 mg/kg, or 2.5-5 mg/kg, e.g., 3-5 mg/kg. For instance, any one of Methods 1-13 or 1.1-1.13, wherein the diffusion enhancing compound is trans-crocetin, in free or pharmaceutically acceptable salt form (e.g., trans sodium crocetinate (TSC)), and is administered at a dose of 0.05-5 mg/kg, e.g., 0.05-2.5 mg/kg (e.g., 1-2.5 mg/kg or 1.5-2.5 mg/kg or 0.5 mg/kg or 1 mg/kg or 1.5 mg/kg or 2 mg/kg or 2.5 mg/kg), e.g., 2-2.5 mg/kg, or 2.5-5 mg/kg, e.g., 3-5 mg/kg.
1.15. Any one of Methods 1-13 or 1.1-1.14, wherein the diffusion enhancing compound is TSC and is administered 4 times per day.
1.16. Any one of Methods 1-13 or 1.1-1.15, wherein the diffusion enhancing compound is TSC and is administered every 6 hours.
1.17. Any one of Methods 1-13 or 1.1-1.16, wherein the diffusion enhancing compound is TSC and is administered for 1-2 days.
1.18. Any one of Methods 1-11 or 1.1-1.17, wherein the disease or condition (e.g., the disease or condition characterized by hypoxia) is ischemia, ischemic or hemorrhagic stroke, traumatic brain injury, respiratory disease, hemorrhagic shock, cardiovascular disease (including peripheral vascular disease), peripheral artery disease, multiple organ failure, atherosclerosis, emphysema, asthma, hypertension, cerebral edema, spinal cord injury, anemia, embolism, thrombosis, myocardial infarction, blood clots, congestive heart failure, acute lung injury, chronic obstructive pulmonary disease, acute respiratory distress syndrome, or wound healing.
1.19. Any one of Methods 1-10, 12, or 1.1-1.17, wherein the disease or condition is cancer and the diffusion enhancing compound is administered in combination with radiation therapy (e.g., prior to, during, or after administration of radiation therapy, e.g., prior to radiation therapy). For instance, wherein the diffusion enhancing compound is administered 30-120 minutes (e.g., 45-60 minutes) prior to administration of radiation therapy.
1.20. Any one of Methods 1-10, 12, 1.1-1.17, or 1.19, wherein the disease or condition is cancer and the diffusion enhancing compound is administered in combination with chemotherapy (e.g., prior to, during, or after administration of chemotherapy, e.g., prior to chemotherapy). For instance, wherein the diffusion enhancing compound is administered 30-120 minutes (e.g., 45-60 minutes or 1-2 hours) prior to administration of chemotherapy.
1.21. Methods 1.19 or 1.20, wherein the cancer is selected from the group consisting of gliomas, glioblastomas, squamous cell carcinomas, melanomas, lymphomas, sarcomas, sarcoids, osteosarcomas, skin cancer, breast cancer, head and neck cancer, gynecological cancer, urological and male genital cancer, bladder cancer, prostate cancer, bone cancer, cancers of the endocrine glands, cancers of the alimentary canal, cancers of the major digestive glands/organs, CNS cancer, lung cancer, brain cancer and pancreatic cancer. For instance, Methods 1.19 or 1.20, wherein the cancer is brain cancer or pancreatic cancer.
1.22. Methods 1.19 or 1.20, wherein the cancer is a solid tumor.
1.23. Methods 1.19 or 1.20, wherein the cancer is a glioma.
1.24. Methods 1.19 or 1.20, wherein the cancer is a glioblastoma multiforme. For instance, Methods 1.19 or 1.20, wherein the cancer is a glioblastoma multiforme and is considered inoperable. For instance, Methods 1.19 or 1.20, wherein the cancer is glioblastoma and the patient is biopsy-only (e.g., there is no resection of the glioblastoma).
1.25. Any one of Methods 1-11 or 1.1-1.17, wherein the disease or condition is ischemia/reperfusion injury.
1.26. Any one of Methods 1-10 or 1.1-1.17, wherein the disease or condition is segment elevation myocardial infarction.
1.27. Any one of Methods 1-10 or 1.1-1.17, wherein the disease or condition is an ischemic wound.
1.28. Any one of Methods 1-13 or 1.1-1.27, wherein the patient is human.
1.29. Any one of Methods 1-13 or 1.1-1.28, wherein the improvement (e.g., increase) by the diffusion enhancing compound (e.g., TSC) is measured by one or both of the SpO₂:FiO₂ (S:F) ratio (e.g., as measured by continuous pulse oximetry) and PaO₂/FiO₂ (P:F) ratio. Or any one of Methods 1-13 or 1.1-1.28, wherein the improvement (e.g., increase) by the diffusion enhancing compound is measured by transcutaneous oximetry measurements (tcpO₂) or SpO₂.
1.30. Any one of Methods 1-13 or 1.1-1.29, wherein the patient achieves one or more endpoints selected from primary and secondary endpoints described in any of Examples 1, 2, or 3 below.
1.31. Any one of Methods 1-13 or 1.1-1.30, wherein the diffusion enhancing compound is TSC and is administered by a continuous intravenous infusion.
1.32. Any one of Methods 1-13 or 1.1-1.30, wherein the diffusion enhancing compound is TSC and is administered by a bolus injection.
1.33. Any one of Methods 1-13 or 1.1-1.32, wherein the total dose (e.g., total daily dose) of the TSC does not result in a visual disturbance (e.g., a yellow visual disturbance).

Further provided is a diffusion enhancing compound (e.g., a bipolar trans carotenoid salt (e.g., TSC)), e.g., as described in any one of Methods 1-13 or 1.1-1.33, for use in any one of Methods 1-13 or 1.1-1.33.

Further provided is use of a diffusion enhancing compound (e.g., a bipolar trans carotenoid salt (e.g., TSC)), e.g., as described in any one of Methods 1-13 or 1.1-1.33, in the manufacture of a medicament for any one of Methods 1-13 or 1.1-1.33.

Further provided is a pharmaceutical composition comprising an effective amount of a diffusion enhancing compound (e.g., a bipolar trans carotenoid salt (e.g., TSC)), e.g., as described in any one of Methods 1-13 or 1.1-1.33, for use in any one of Methods 1-13 or 1.1-1.33.

### Example 1

| | |
|---|---|
| Title of Study | Randomized, double-blind, placebo-controlled, pharmacokinetic, |
| | pharmacodynamic study of Trans Sodium Crocetinate utilizing |
| | Transcutaneous Oximetry Measurement in healthy volunteers |
| Study | Determine the dose response of Trans Sodium Crocetinate (TSC) on |
| Objectives | Transcutaneous Oximetry Measurements (tcpO₂) following a single |
| | administration of TSC in subjects breathing 100% oxygen (O₂) |
| Study | Healthy volunteers aged 18-55 |
| Population | |
| Participant | Maximum of 13 days, inclusive of screening, treatment, and follow up |
| Duration | |
| Eligibility | Inclusion Criteria |
| | 1. Healthy male or female, age 18-55 |
| | 2. Able and willing to lie quietly supine or semi-recumbent for up to 2.5 hours |
| | 3. Abstinence from exercise, caffeine, alcohol, and a heavy meal on the day of |
| | the treatment visit |
| | 4. Subject is able to communicate effectively with the Investigator and to |
| | comply with all study requirements, restrictions, and directions from the study |
| | staff |
| | Exclusion Criteria |
| | 1. Allergy to study medication |
| | 2. Pregnant or lactating |
| | 3. Current smoker |
| | 4. Body Mass Index (BMI) >30 |
| | 5. Any skin condition on limbs to be tested that could impair testing (rash, |
| | wound, prior radiation therapy, other skin conditions, per Principal Investigator |
| | (PI) discretion) |
| | 6. Known cardiovascular disease, including treated or untreated hypertension |
| | 7. Significant respiratory disease and/or any other significant medical condition |
| | 8. Subject has an acute illness (gastrointestinal, influenza, or known |
| | inflammatory process) at the Treatment Visit |
| | 9. Urine screen positive for drugs or alcohol (at screening and enrollment) |
| | 10. Concomitant medications used to treat a diagnosed medical condition |
| | 11. Subject who, for any reason, is deemed by the Investigator to be unsuitable |
| | for the study; or has any condition that would interfere with the evaluation of |
| | tissue oxygen measurements or PK of the investigational drug; or is otherwise |
| | unable to comply with the protocol |
| Safety | Assessment of adverse events, new medications, laboratory (complete |
| | blood count [CBC], basic metabolic panel [BMP]), vital signs (blood |
| | pressure, heart rate, respiratory rate), and oxygen saturation (SpO₂) |
| Study Design | Randomized, double-blind, placebo-controlled, pharmacokinetic, |
| | pharmacodynamic study |
| | Subjects are randomized to a single IV bolus dose of TSC (0.5, |
| | 1.0, 1.5, 2.0, or 2.5 mg/kg) or placebo (normal saline). |
| Study | Subjects are randomized into one of 6 groups in a 1:1:1:1:1:1 schema, to include |
| Overview | the 5 TSC doses (based on screening body weight) and placebo normal saline (7 |
| | mL dose). To maintain the double-blind, study drug administration is performed |
| | by unblinded medical staff who are not be involved in other study procedures, |
| | including subject assessment. Subjects, investigators and study coordinators do |
| | not see the injection or injection site or be aware of randomization. |
| | On the day of treatment, subjects are maintained in a temperature controlled |
| | room (between 22.0 and 25.0° C.), and in a supine position with the head slightly |
| | raised on one pillow, or semi-recumbent. One blanket is provided for comfort. |
| | An IV catheter is placed for study drug administration and PK measurement. |
| | TcpO₂ sensor electrodes are applied to the left or right lower extremity, per PI |
| | discretion. Sensor electrode temperature is preset to 45° C., to allow maximum |
| | vasodilation. Risk of sensor site superficial burn is minimal given the relatively |
| | brief testing period. Four (4) sensors are applied to the following locations: |
| | Sensor 1: Mid-dorsum of the foot |
| | Sensor 2: 10 cm distal to the lateral femoral epicondyle |
| | Sensor 3: 5 cm proximal to the anterior aspect of the lateral malleolus |
| | Sensor 4: 5 cm proximal from the center of the medial malleolus |
| | After the topO₂ sensors have been applied and tested, subjects are placed on O₂ |
| | via a simple facemask at 6 L/minute, and remain on O₂ for 70 minutes prior to |
| | study drug administration. The first 10 minutes allow for equilibration of O₂ |
| | levels, and the subsequent 60 minutes serve as the baseline period. TcpO₂ |
| | values and SpO₂ are recorded every 5 minutes during the above periods. At the |
| | end of the 70-minute equilibration/baseline period, subjects receive a single IV |
| | bolus injection of TSC at a dose of 0.5, 1.0, 1.5, 2.0 or 2.5 mg/kg, or placebo. |
| | After study drug has been administered, subjects continue on O₂ and are |
| | evaluated for an additional 60 minutes, with tcpO₂ values and SpO₂ recorded |
| | every 5 minutes. |
| | In addition to assessment of tcpO₂ every 5 minutes, continuous tcpO₂ |
| | measurements are provided by the TCOM machine in graphical format. |
| | Vital signs are assessed prior to study drug dosing, and at 10, 30, and 60 |
| | minutes post-dosing. Adverse events are assessed throughout. |
| | Prior to and following study drug administration, PK samples are obtained at |
| | the below intervals. |
| | Pre-dose (within 10 minutes prior to injection) |
| | 1 minute post end of injection (+1 minute) |
| | 10 minutes post end of injection (±1 minute) |
| | 30 minutes post end of injection (±1 minute) |
| | 1.5 hours post end of injection (±2 minutes) |
| | After the 60 minute post-treatment evaluation period, oxygen is discontinued |
| | and the tcpO₂ sensor electrodes removed. Subjects remain in clinic for an |
| | additional 60 minutes to allow for collection of the 1.5-hour PK blood draw, |
| | and observation prior to discharge. Vital signs re repeated at discharge. |
| | Subjects are contacted by telephone at 48 hours (+2 days) for a safety follow up |
| | to assess adverse events and new medications. |
| Statistical | Subjects re randomized using a non-stratified permuted block randomization |
| Analysis Plan | scheme. |
| | The primary analysis is based on the change over time in the tcpO₂ |
| | measurements following a single administration of TSC in subjects breathing |
| | 100% O₂. The time-matched changes from Period 1 (60 minute run-in on 100% |
| | O₂) to Period 2 (60 minute post-drug administration on 100% O₂) serve as the |
| | dependent variable in the repeated measures analysis. The 60 minute run-in on |
| | 100% O₂ is intended to account for the intra-subject variability over time and |
| | the time-match differences represent the least biased estimate of the effect of the |
| | study drug. |
| | Adverse events are coded and summarized by system organ class and preferred |
| | term and presented by randomized treatment assignment, Period (1 or 2), and |
| | overall, independent of Period. |

### Study Rationale

Transcutaneous Oximetry (tcpO₂) is a well characterized and a non-invasive method of measuring the partial pressure of oxygen (oxygen tension) under the skin. This is different from pulse oximetry which uses infrared technology to measure hemoglobin saturation in the blood (not in the tissues). TSC's unique mechanism of action is to enhance diffusion of oxygen, and tcpO₂ methodology uniquely measures the amount of oxygen in the tissues that has diffused from the microcirculation.

This trial measures tcpO₂ in multiple locations on the lower extremity of a healthy volunteer after a single dose of TSC.

### Study Methods

### Study Overview

This is a randomized, double-blind, placebo-controlled, pharmacokinetic, pharmacodynamics study of Trans Sodium Crocetinate (TSC) utilizing Transcutaneous Oximetry Measurement (tcpO₂) in healthy volunteers breathing 100% O₂. Study assessments include tcpO₂ levels, SpO₂, and PK.

### Study Objectives

### Primary Endpoint

Determine the dose-response of TSC on tcpO₂ following a single administration of TSC in subjects breathing 100% O₂.

### Study Population

Subjects are healthy adult volunteers meeting all inclusion and exclusion criteria. Subjects are randomized to a single IV bolus injection of TSC at a dose of 0.5, 1.0. 1.5, 2.0, or 2.5 mg/kg or placebo normal saline with concomitant O₂ administration.

Thirty subjects are in the study with a mean age of 33 (±9 years) and include non-smokers, distinct ethnicities/race, and a balanced ratio of males and females. One subject in the 2.5 mg/kg dose cohort experiences inadvertent subcutaneous infiltration of TSC due to IV cannula migration and is excluded from the analysis. Study results reflect pharmacokinetic and pharmacodynamics analyses of 24 and 29 subjects, respectively.

### Randomization

Volunteers are randomized to one of 5 TSC doses or placebo in a 1:1:1:1:1:1 schema, with 5 subjects randomized to each of the 6 cohorts (30 subjects total).

### Dosing Regimen

Drug is administered as a one-time IV bolus injection to 5 unique subjects per dose cohort, to include 5 TSC dose levels and placebo. Placebo consists of 7 mL of normal saline.

### Treatment Visit (Day 0)

Following the screening visit, the subject returns to the clinic for study treatment procedures. If the site has Point of Care (POC) laboratory testing available, the screening and treatment procedures may occur on the same day. Subjects refrain from exercise, caffeine, alcohol, and a heavy meal on the day of the treatment visit.

Subjects are randomized into one of 6 groups in a 1:1:1:1:1:1 schema, to include the 5 TSC doses (based on screening body weight) and placebo normal saline (7 mL dose). To maintain the double-blind, study drug administration is performed by unblinded medical staff who are not be involved in other study procedures, including subject assessment. Subjects, investigators, and study coordinators do not see the injection or injection site or be aware of randomization.

### Subject Arrival and TcpO₂ Sensor Electrode Placement

When subjects arrive to clinic for the treatment visit, medical history, concomitant medications, urine pregnancy screen (female of childbearing potential), urine drug screen, and alcohol screen are assessed (unless the Screening and Treatment visits are done on the same day).

Subjects are maintained in a temperature-controlled room (between 22.0 and 25.0° C.), and in a supine position with the head slightly raised on one pillow, or semi-recumbent. One blanket is provided for comfort. An IV catheter is placed for study drug administration and PK measurement.

TcpO₂ sensor electrodes are applied to the left or right lower extremity, per PI discretion. Sensor electrode temperature is preset to 45° C., to allow maximum vasodilation. Risk of sensor site superficial burn is minimal given the relatively brief testing period. Four (4) sensors are applied to the following locations:
Sensor 1: Mid-dorsum of the foot
Sensor 2: 10 cm distal to the lateral femoral epicondyle
Sensor 3: 5 cm proximal to the anterior aspect of the lateral malleolus
Sensor 4: 5 cm proximal from the center of the medial malleolus

### O₂ Equilibration and Baseline Period

After the tcpO₂ sensors have been applied and tested, subjects are placed on O₂ via a simple facemask at 6 L/minute, and remain on O₂ for 70 minutes prior to study drug administration. The first 10 minutes allow for equilibration of O₂ levels, and the subsequent 60 minutes serve as the baseline period. TcpO₂ values and SpO₂ are recorded every 5 minutes during the above periods. Baseline vital signs are measured within 10 minutes prior to study drug dosing.

Once the equilibration phase begins, it is important that subjects remain lying quietly and minimize bodily movement through the entire baseline and treatment periods.

### Treatment Evaluation Period

At the end of the 70-minute equilibration/baseline period, subjects continue on O₂ and receive a single IV bolus injection of TSC at a dose of 0.5, 1.0, 1.5, 2.0 or 2.5 mg/kg, or placebo. After study drug administration, subjects are evaluated for 60 minutes, with tcpO₂ values and SpO₂ recorded every 5 minutes. In addition to assessment of tcpO₂ every 5 minutes, continuous tcpO₂ measurements are provided by the TCOM machine in graphical format.

Vital signs during the treatment and evaluation period are assessed at 10, 30, and 60 minutes post-study drug dosing. Adverse events are assessed throughout.

### PK Measurements

Prior to and following study drug administration, PK samples are obtained at the below intervals:
Pre-dose (within 10 minutes prior to injection)
1 minute post end of injection (+1 minute)
10 minutes post end of injection (±1 minute)
30 minutes post end of injection (±1 minute)
1.5 hours post end of injection (±2 minutes)

Plasma is assayed for TSC using LC-MS/MS using prednisone as an internal standard. Sample pre-treatment involves a protein precipitation extraction procedure. Lower limit of quantitation is 10 ng/ml.

For 19 of the 24 subjects included in the PK analysis, TSC concentration (Cp: Concentration in Plasma.) in the 1-minute sample is smaller (often many-fold) compared to the 10-minute sample, most likely a result of sampling too soon after the dose is administered. As a result, all 1-minute samples are excluded from population PK analysis.

Based on previous analyses and examination of drug concentration profiles, a two-compartment model with no covariates fit the data well. To determine whether the systemic parameters should be scaled for body size, three scaling approaches are evaluated: scaling all parameters by weight, allometric scaling (clearances scaled by weight raised to the 0.75 power; distribution volumes scaled by weight), and scaling all parameters by weight raised to an estimated power. The weight normalized model is preferred statistically (evaluated by the decrease in the objective function [similar to a sum of squares]) and by graphics. Graphics suggest that clearance varied as a function of dose. A model is evaluated in which clearance varied as a linear function of dose:

$$CL = ( 1 - THETA * ( DOSE / 1.5 ) \cdot TVCL \cdot EXP ( ETA )$$

where CL, each subject's individual (post hoc) value for clearance, is the product of linear function of dose (where DOSE is the dose in mg/kg and THETA is estimated), 1.5 is the median dose (thereby centering the model), TVCL is the “typical” value for clearance (which applies to subjects receiving the 1.5 mg/kg dose, and EXP(ETA) is a term that allows for inter-individual variability. Pharmacokinetic parameters are displayed in Table 1.

TABLE 1
Population Pharmacokinetic Parameters of TSC

| Description | Estimate | Inter-individual Variability* |
|---|---|---|
| SCALE | (WT/75)† | |
| FCTR | 1-0.53677 · (DOSE§/1.5) | —¶ |
| Clearance (L/day) | 196.561 · SCALE · FCTR | 0.6189 |
| V₁ (L) | 4.27707 · SCALE | 0.1869 |
| Distribution Clearance (L/day) | 161.176 · SCALE | 0.1296 |
| V₂ (L) | 46.61 · SCALE | 0.6743 |

*Quantified as sqrt(OMEGA²) where OMEGA² is the variance of inter-individual variability.
†WT is weight in kg; 75 kg is the median weight in the study
§DOSE is dose in mg/kg; 1.5 is the median value
¶Inter-individual variability is not permitted for this term.

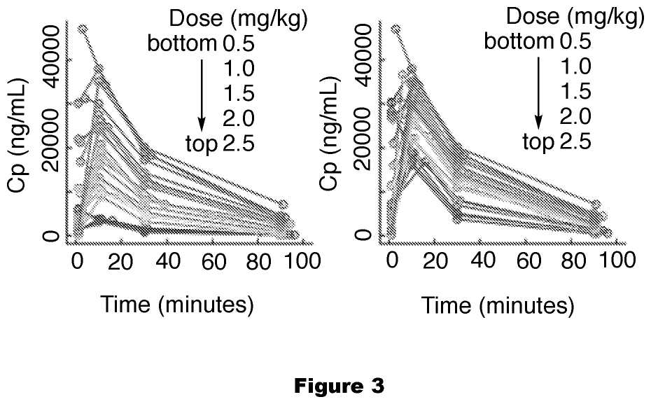

TSC plasma concentrations increase with escalating dose, with the 2.5 mg/kg dose administration leading to the highest plasma concentration. Raw values are presented in the left panel in FIG. 3 and concentrations normalized to a TSC dose of 2.5 mg/kg are presented in the right panel in FIG. 3.

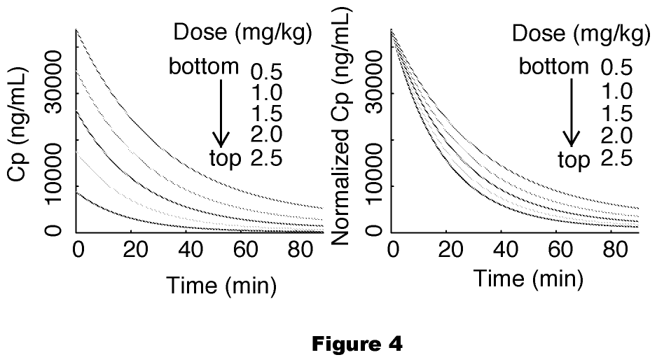

The concentration profile for a hypothetical subject weighing 75 kg administered escalating drug doses from 0.5-2.5 mg/kg is in FIG. 4. In the right panel in FIG. 4, concentrations are normalized to a dose of 2.5 mg/kg to illustrate the non-linearity with respect to dose.

### Post-Treatment Evaluation Period

After the 60-minute treatment evaluation period, the tcpO₂ sensor electrodes are removed. Subjects remain in clinic for an additional 60 minutes to allow for collection of the 1.5-hour PK blood draw, and observation prior to discharge. Vital signs are repeated at discharge.

### Laboratory

Clinical laboratory evaluations:
Fasting is not required before collection of laboratory samples
Blood is collected at the time points indicated in the protocol

### Primary and Secondary Efficacy Analyses

The primary analysis is based on the change over time in the tcpO₂ measurements following a single administration of TSC in subjects breathing 100% O₂. The time-matched changes from Period 1 (60 minute run-in on 100% O₂) to Period 2 (60 minute post-drug administration on 100% O₂) serve as the dependent variable in the repeated measures analysis. The 60 minute run-in on 100% O₂ is intended to account for the intra-subject variability over time and the time-match differences represent the least biased estimate of the effect of the study drug.

### Pharmacodynamic Analysis of Peripheral Tissue Oxygenation

The overall pooled median values across the four TcpO₂ sensors are calculated using the median value from each sensor during the same interval. To compare the time-matched changes in TcpO₂, a 2-factor (treatment and time) repeated measures (time) analysis of variance (ANOVA) model is used. Contrast statements within the model facilitate comparisons between individual dose arms and placebo over time. Intra-subject changes (Period 2 minus Period 1 in 5-minute intervals) serve as the dependent variable in the model. TcpO₂ baseline measures during the 60-minute Period 1 are adjusted for intra-subject variability over time. Time-matched differences between values obtained during Period 1 and Period 2 represent the least biased estimate of the effect of the study drug versus placebo.

### Supplemental Analysis of Tissue Oxygenation Pharmacodynamics

The raw data recordings of the individual TcpO₂ sensor measurements demonstrate unexpected variability among sensors, suggesting that the data should not be pooled across all four sensors. Therefore, a supplementary pharmacodynamic analysis on the intra-subject slopes from each period and sensor is performed. For Period 1, on an intra-subject basis, the median value from each of the 12 intervals is used to construct the slope using orthogonal spacing with respect to time. For Period 2, the slope is constructed using the median value from each of the 13 intervals—of note, Period 2 observations include 1- and 2-minute values—in addition to the 5-minute measurements for comparison to the 12 corresponding intervals of Period 1. An additional set of analyses are conducted using the Period 2 intra-subject slopes as the dependent variable. The active dose groups are pooled and the least square means are compared to placebo. This procedure is repeated iteratively, each time removing the next lowest dose group to determine the separation of each cohort's least square means relative to placebo.

### Results

TSC is safe and well tolerated at all doses tested. The pharmacokinetic analysis demonstrates that clearance decreases at escalating doses of TSC. The results of the primary pharmacodynamic analysis reveal high levels of variability in the 60-minute baseline TcpO₂ levels, however despite such variability, time-matched TcpO₂ measurements demonstrate observed increases in median TcpO₂ values in subjects who received TSC, relative to those who receive a placebo. The high variability observed across the four sensors suggest that the data should not be pooled across all four sensors; therefore, additional supplemental analyses are performed. The results of the supplemental analysis indicate that the TcpO₂ intra-subject slopes of the TSC treatment groups are consistently positive during the study intervention period, and therefore suggestive of an increase in TcpO₂ levels. This is not observed in the placebo group. Based on this analysis, there is an observed dose effect where all TSC dose groups have a greater increase in TcpO₂ levels than the placebo group, with the 2.5 mg/kg dose demonstrating the most notable increase over the 1-hour intervention period (Period 2).

Transcutaenous oxygen monitoring (TCOM) is used to measure the direct pharmacodynamic effects of TSC on peripheral tissue oxygenation. Results show a positive trend in TCOM readings after TSC administration compared to placebo that persists up to 60 minutes. The effects are most pronounced at the higher doses (2.0 mg/kg and 2.5 mg/kg iv). TSC is safe and well-tolerated with no serious adverse events or dose-limiting toxicities up to and including the 2.5 mg/kg iv dose.

### Pharmacodynamics

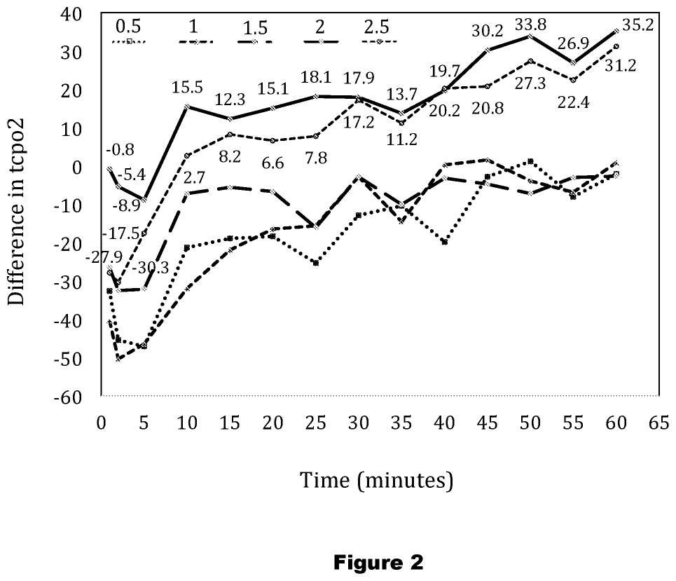

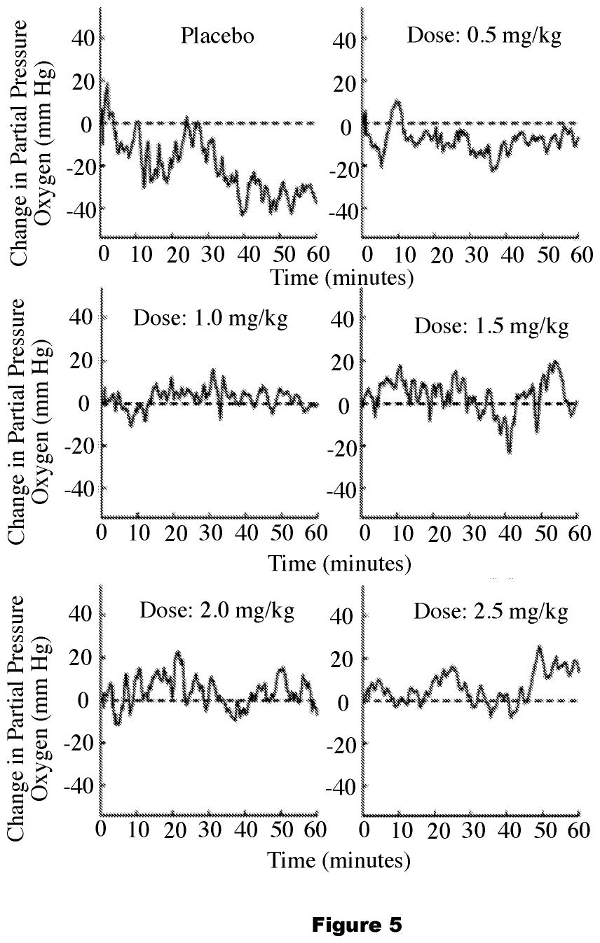

Baseline TcpO₂ values recorded in this study are between 150-200 mmHg which is in line with previous studies of healthy individuals. The time-matched TcpO₂ values reporting changes in the amount of oxygen in tissue extremities fluctuate during baseline Period 1, an unexpected finding. The high variability in the baseline Period 1 TcpO₂ readings do not clearly demonstrate time matching trends; however, the placebo group displays a time related decrease in peripheral oxygenation during Period 2. Conversely, the TSC groups display stable or increasing peripheral oxygenation levels over time (FIG. 5). FIG. 5 shows changes from baseline in the median transcutaneous oxygen tension values across all dose levels. A time related decrease in peripheral oxygenation in the placebo group is observed and in contrast stable peripheral oxygenation levels over time in TSC treated groups. Furthermore, Period 2 assessments of the difference in tcpO₂ values between TSC and placebo (FIG. 2) suggest an increase in peripheral TcpO₂ values following TSC treatment in the subject groups administered higher TSC doses (≥2 mg/kg). The subtraction of the placebo TcpO₂ levels from the TSC treated groups in Period 2 illustrates an increase in TcpO₂ values post TSC administration relative to placebo in the higher TSC dose groups (≥2 mg/kg). For Period 2, the median value is used from each of the 13 intervals of 1, 3- and 5-minute duration.

The unexpected range of variability observed in the 60-minute baseline portion (Period 1) among the four sensors suggest that the data should not be pooled across all four sensors. Therefore, additional supplemental analyses are performed as described above. The supplemental analysis demonstrate that the subjects of the TSC groups have consistently positive intra-subject slopes across multiple sensors observed during Period 2 that are not observed for the placebo group (Table 2). In addition, pooling the TSC groups and comparing their least square means to those of the placebo arm within Period 2 reveals a significant separation across all four sensors (Tables 3-6). The greatest least square mean difference occurs with 2.5 mg/kg dose versus to placebo.

Differences are observed in sensor 1 which has a greater number of positive intra-subject slopes and increased least square means compared to the other three sensors (Tables 2 and 3). However, the results of sensor 1 are similar to those of the other sensors, in which the TSC treated groups display increases in TcpO₂ slopes which are not observed in the placebo group. Furthermore, sensor 1 is the most distal sensor in all subjects (placed on the mid-dorsum of the foot) which may in part explain the larger observed change in the TcpO₂ levels at this sensor's anatomic location. Augmentation in peripheral oxygenation would potentially be most notable when starting from a lower baseline level of tissue oxygenation which can occur in the most distal parts of the body, and it is conceivable that more significant increases in TcpO₂ values would be observed in patient populations who are hypoxic at baseline.

Overall, the supplemental analysis is suggestive of an effect across all sensors in which the TSC groups have a greater increase in TcpO₂ than placebo. Of note, the 2.5 mg/kg dose results in the greatest difference from placebo over the 1-hour treatment period (Period 2). There is no correction for multiplicity across the analyses, given the probability values are calculated for informational purposes.

TABLE 2
Number (%) of Subjects with a TcpO₂ increase
during Period 2, Treatment vs. Placebo

| | TSC-treated | Placebo-treated |
|---|---|---|
| Sensor Number | Subjects (N = 24) | Subjects (N = 5) |
| 1 | 16/24 (66.7%) | 1/5 (20.0%) |
| 2 | 4/24 (16.7%) | 0/5 (0.0%) |
| 3 | 3/24 (12.5%) | 0/5 (0.0%) |
| 4 | 6/24 (25.0%) | 0/5 (0.0%) |

As shown in Table 2, in all sensors TSC treated subjects display positive slopes which is not the case in the placebo treated subjects (with the exception of one subject in sensor 1). Sensor 1 differs from the others with respect to the number of subjects that have a significant slope during Period 2, independent of the treatment assignment. One subject (assigned 2.5 mg/kg) receives an inadvertent subcutaneous injection of TSC due to IV cannula malposition and is excluded from both the Pharmacokinetic and Pharmacodynamic analysis. As a result, the pharmacodynamic analyses includes 29 subjects.

TABLE 3
Summary of the Intra-Subject Slopes from Sensor 1 during
Period 1 and 2 by Randomized Treatment Assignment

| | Groups | Least | |
|---|---|---|---|
| | being | Square | Probability |
| Sensor 1 | Compared | Mean | Value |
| Pooling all TSC doses vs. placebo | TSC | 0.3185 | 0.0876 |
| | Placebo | −0.1878 | |
| Pooling TSC doses ≥1.0 mg/kg | TSC | 0.3537 | 0.0960 |
| vs. placebo | Placebo | −0.1878 | |
| Pooling TSC doses ≥1.5 mg/kg | TSC | 0.3027 | 0.1702 |
| vs. placebo | Placebo | −0.1878 | |
| Pooling TSC doses ≥2.0 mg/kg | TSC | 0.4359 | 0.1430 |
| vs. placebo | Placebo | −0.1878 | |

As shown in Table 3, differences between the least square means of the placebo group and the TSC dose groups reveal that TSC groups have a greater trajectory than the placebo group.

TABLE 4
Summary of the Intra-Subject Slopes from Sensor 2 during
Period 1 and 2 by Randomized Treatment Assignment

| | Groups | Least | |
|---|---|---|---|
| | being | Square | Probability |
| Sensor 2 | Compared | Mean | Value |
| Pooling all TSC doses vs. placebo | TSC | 0.0849 | 0.0796 |
| | Placebo | −0.3481 | |
| Pooling TSC doses ≥1.0 mg/kg | TSC | 0.0656 | 0.1139 |
| vs. placebo | Placebo | −0.3481 | |
| Pooling TSC doses ≥1.5 mg/kg | TSC | 0.0149 | 0.1306 |
| vs. placebo | Placebo | −0.3481 | |
| Pooling TSC doses ≥2.0 mg/kg | TSC | 0.0753 | 0.0959 |
| vs. placebo | Placebo | −0.3481 | |

As shown in Table 4, differences between the least square means of the placebo group and the TSC dose groups reveal that TSC groups have a greater trajectory than the placebo group.

TABLE 5
Summary of the Intra-Subject Slopes from Sensor 3 during
Period 1 and 2 by Randomized Treatment Assignment

| | Groups | Least | |
|---|---|---|---|
| | being | Square | Probability |
| Sensor 3 | Compared | Mean | Value |
| Pooling all TSC doses vs. placebo | TSC | 0.0151 | 0.0186 |
| | Placebo | −0.6320 | |
| Pooling TSC doses ≥1.0 mg/kg | TSC | 0.0132 | 0.0301 |
| vs. placebo | Placebo | −0.6320 | |
| Pooling TSC doses ≥1.5 mg/kg | TSC | 0.0249 | 0.0572 |
| vs. placebo | Placebo | −0.6320 | |
| Pooling TSC doses ≥2.0 mg/kg | TSC | 0.0846 | 0.0421 |
| vs. placebo | Placebo | −0.6320 | |

As shown in Table 5, differences between the least square means of the placebo group and the TSC dose groups reveal that TSC groups have a greater trajectory than the placebo group. These differences are significant (p value≤0.05) when pooling all TSC doses, TSC doses≥1.0 mg/kg and TSC doses≥2.0 mg/kg.

TABLE 6
Summary of the Intra-Subject Slopes from Sensor 4 during
Period 1 and 2 by Randomized Treatment Assignment

| | Groups | Least | |
|---|---|---|---|
| | being | Square | Probability |
| Sensor 4 | Compared | Mean | Value |
| Pooling all TSC doses vs. placebo | TSC | −0.0198 | 0.0230 |
| | Placebo | −0.6389 | |
| Pooling TSC doses ≥1.0 mg/kg | TSC | 0.0260 | 0.0213 |
| vs. placebo | Placebo | −0.6389 | |
| Pooling TSC doses ≥1.5 mg/kg | TSC | −0.0909 | 0.0436 |
| vs. placebo | Placebo | −0.6389 | |
| Pooling TSC doses ≥2.0 mg/kg | TSC | 0.0297 | 0.0187 |
| vs. placebo | Placebo | −0.6389 | |

As shown in Table 6, differences between the least square means of the placebo group and the TSC dose groups reveals that TSC groups have a greater trajectory than the placebo group. These differences are significant (p value≤0.05) in all groups.

### Safety

TSC is safe and well tolerated, with a total of nine treatment-emergent adverse events reported overall among seven of the 30 subjects (23.3%). Of the nine treatment-emergent adverse events experienced by five subjects (16.7%), five are determined to be drug-related and all are deemed mild in intensity. The most frequent treatment-emergent adverse event is post procedural erythema experienced by two subjects; others include transient chromaturia, post procedural pruritus, headache, taste disorder, injection site pain, and injection site streaking. No subjects experience a serious adverse event or withdraw from the study for any reason.

### CONCLUSIONS

TSC administered as a single IV bolus dose ranging from 0.5 mg/kg to 2.5 mg/kg to healthy subjects while breathing supplemental oxygen, is safe and well tolerated. Pharmacokinetic assessments demonstrate that TSC plasma concentrations increase with escalating dose and that increasing TSC dose is associated with a decrease in clearance. The high levels of variability in TcpO₂ levels do not allow for pooling of sensor measurements for primary analysis; however, supplemental analysis of individual sensor measurements demonstrate an observed dose effect of TSC on peripheral tissue oxygenation relative to placebo.

The results of the pharmacodynamic analysis display a time related decrease in peripheral oxygenation during Period 2 in the placebo group which is not observed in the TSC groups. Hypotheses for this observation might include: reduction in minute ventilation and/or reduction in cardiac output in the second hour with subjects being in a semi-recumbent position and relaxing conditions. Importantly the TSC treated groups treated in identical conditions do not display such a decrease in peripheral oxygenation across all TSC doses. In addition, the results of the supplementary pharmacodynamic analysis on TcpO₂ measurements during Period 2 reveal increases in median TcpO₂ values in subjects who received TSC compared to placebo.

The observed variability across individual TcpO₂ sensors that do not allow for pooling of sensor readings for a direct time-matched comparison. Therefore, a sensor-by-sensor supplemental pharmacodynamic analysis is performed, first calculating the slope of median values of intra-subject TcpO₂ interval measures across individual sensors during Period 1, then constructing intra-subject slopes using the TcpO₂ interval measures of Period 2. Additional analysis of the Period 2 intra-subject, individual sensor slopes to the least square means of the active dose cohorts-versus-placebo calculations demonstrate consistent positive increases in TcpO₂ readings across multiple individual sensors in Period 2 for all TSC groups when compared to the placebo group. The greatest TcpO₂ difference is observed when the 2.5 mg/kg TSC group is compared to the placebo group, suggesting that the effect of TSC is greatest at higher doses.

### Example 2

### Altitude Protocol Synopsis

| | |
|---|---|
| Title of Study | Randomized, double-blind, placebo controlled, crossover study of Trans |
| | Sodium Crocetinate in healthy volunteers exercising at altitude |
| Study | The Primary Objective is to determine the effect of Trans Sodium Crocetinate |
| Objectives | (TSC) on partial pressure of oxygen (PaO₂) and maximal oxygen consumption |
| | (VO₂ max) in healthy volunteers exercising at altitude. |
| | The Secondary Objective is to assess the effect of TSC on oxygen saturation |
| | (SpO₂) and lactate in healthy volunteers exercising at altitude. |
| Endpoints | Primary endpoints will be 1) comparison of PaO₂ at altitude between control |
| | and experimental exposures; and 2) comparison of VO₂ max at altitude between |
| | control and experimental exposures. |
| | Secondary endpoints will include a comparison of oxygen saturation (SpO₂) and |
| | lactate between the control and experimental exposures. |
| Study | Healthy volunteer men and women age 18-40, capable of signing their own |
| Population | informed consent, will be enrolled in this study. Special populations such as |
| | pregnant women, prisoners, and children will not be enrolled in the study. |
| Participant | Maximum of 32 days, inclusive of screening, treatment, and follow up |
| Duration | |
| Eligibility | Inclusion Criteria |
| | 1. Healthy males and females ages 18-40 at screening |
| | 2. Non-smoking |
| | 3. Able to provide informed consent and agree to adhere to all study visits and |
| | requirements |
| | 4. Females of childbearing potential must have a negative blood pregnancy test |
| | at screening and agree to use one of the accepted contraceptive regimens, or a |
| | double method of birth control during the study and at least 30 days after the |
| | last dose of study |
| | 5. Males who engage in sexual activity that has the risk of pregnancy must |
| | agree to use a double barrier method and agree not to donate sperm during the |
| | study and for at least 90 days after the last dose of study drug |
| | Exclusion Criteria |
| | 1. Allergy to study medication |
| | 2. Pregnant or breast feeding |
| | 3. Received investigational medicine (IMP) within past 30 days |
| | 4. VO₂ max <35 mL/kg/min (male), <30 mL/kg/min (female) at screening |
| | 5. Abnormal pulmonary function testing at screening |
| | 6. Surgery or hospitalization in past 3 months determined by the PI to be |
| | clinically relevant. |
| | 7. History of ongoing alcohol or substance abuse |
| | 8. Known cardiovascular disease, including treated or untreated hypertension |
| | 9. Respiratory disease and/or any other significant medical condition, including |
| | psychiatric disorders per PI discretion |
| | 10. Clinically significant abnormality on ECG per PI discretion |
| | 11. Blood donation (excluding plasma donation) of approximately 500 mL |
| | within 56 days prior to screening |
| | 12. Plasma donation within 7 days prior to screening |
| | 13. Treatment with an investigational drug within 30 days or 5 times the half- |
| | life (whichever is longer) prior to screening |
| | 14. History of smoking |
| | 15. Urine screen positive for drugs or positive breathalyzer for alcohol (at |
| | screening and Treatment Visit Day 1) |
| | 16. History of seizures |
| | 17. Previous pneumothorax or pneumomediastinum |
| | 18. Hypo/Hyperglycemia |
| | 19. Diabetes |
| | 20. Regularly taking medications which may alter heart rate, blood pressure or |
| | cardiac output per PI discretion |
| | 21. Previous history of middle ear equalization problems at discretion of PI |
| Safety | Adverse Events (AE), and Serious Adverse Events (SAE) will be reported per |
| | protocols. Laboratory (complete blood count [CBC], basic metabolic panel |
| | [BMP] HIV, HBsAg, HCVAb), vital signs (blood pressure, heart rate, |
| | respiratory rate, temperature), and oxygen saturation (SpO₂) will be monitored. |
| Study Design | Randomized, double-blind, placebo-controlled, crossover study. |
| | Subjects will be randomized to a single IV bolus dose of TSC (0.5 mg/kg, 1.5 |
| | mg/kg, or 2.5 mg/kg). Each subject will complete the altitude exposure twice in |
| | a random order on the same day (Treatment Visit Day 1), thereby serving as |
| | their own control. One exposure they will receive a single IV dose of TSC, and |
| | the other exposure will receive a single IV dose of normal saline as placebo |
| | prior to exercise at simulated altitude. |
| Study | Randomization & Blinding: |
| Overview | This is a prospective, randomized control, crossover trial. Each subject will |
| | complete the experiment twice in a random order, thereby serving as their own |
| | control. Randomization schedule will be assigned by study statistician 1:1:1 to |
| | TSC dose cohort as well as randomization of exposure sequence on treatment |
| | day. Each TSC dose will be calculated based on the subject's body weight in kg |
| | at the dose they are randomized to. Each individual dose of placebo will be |
| | normal saline 7 mL. |
| | Investigators and subjects will be blind to the randomization schedule unless |
| | circumstances arise affecting the health and safety of subjects. Study drug and |
| | placebo will be dispensed by the pharmacy in a manner preventing |
| | identification of drug/placebo by the investigators or subjects. |
| | Pre-exposure assessment and preparation (Treatment Visit Day 1): |
| | 1. Re-assess Inclusion/Exclusion criteria are met |
| | 2. Urine pregnancy test for WOCBP |
| | 3. Re-check UDS and breathalyzer |
| | 4. Subjects will refrain from exercise, caffeine, alcohol, nicotine, and a heavy |
| | meal prior to testing on the day of the treatment visit |
| | 5. A radial arterial catheter and peripheral IV will be placed and secured prior to |
| | the subject entering the chamber. |
| | 6. Baseline Arterial Blood Gas (ABG) will be analyzed (PaO2, PaCO2, pH, |
| | HCO3, lactate |
| | 7. Baseline vital signs, including temperature, heart rate, blood pressure and |
| | pulse oximetry, will be recorded. |
| | Altitude Exposure #1: |
| | 1. The experiment will be carried out in a chamber at a Hypobaric Center. |
| | 2. Subject will be connected to physiologic monitoring equipment to record 12- |
| | lead ECG, arterial line blood pressure and pulse oximetry. |
| | 3. Subject, medical tender (physician, resident physician, nurse or paramedic) |
| | and research technician will ascend to 15,000 ft altitude per chamber protocol. |
| | 4. Once at 15,000 ft, subject will be monitored for continuously with EKG, |
| | pulse oximetry and blood pressure. |
| | 5. At altitude, pre-exercise blood gas will be collected. |
| | 6. Subject will receive placebo or study drug via intravenous line. |
| | 7. Subject will perform VO₂ max testing on stationary bicycle per site protocol. |
| | 8. Arterial blood gases will be drawn every three minutes during exercise. |
| | 9. Physiologic data will be recorded into software and metabolic cart. |
| | 10. Final arterial blood gas including lactate, will be drawn at completion of |
| | VO₂ max protocol (10 ± 1 minutes post-exercise at altitude). |
| | 11. Chamber will return to sea-level atmospheric pressure. |
| | 12. Subjects will complete a post-exposure survey inquiring about side effects |
| | and perceived performance. |
| | Altitude Exposure #2: |
| | 1. A rest period of at least 2 hours outside of the chamber will take place |
| | between the two altitude exposures. |
| | 2. Subject will again be connected to physiologic monitoring equipment to |
| | record 12-lead ECG, arterial line blood pressure and pulse oximetry. |
| | 3. Subject, medical tender (physician, resident physician, nurse or paramedic) |
| | and research technician will ascend to 15,000 ft altitude per chamber protocol. |
| | 4. Once at 15,000 ft, subject will be monitored for continuously with EKG, |
| | pulse oximetry and blood pressure. |
| | 5. Altitude, pre-exercise blood gas will be collected. |
| | 6. Subject will receive placebo or study drug via intravenous line. |
| | 7. Subject will perform VO₂ max testing on stationary bicycle. |
| | 8. Arterial blood gases will be drawn every 3 minutes during exercise. |
| | 9. Physiologic data will be recorded into software and metabolic cart. |
| | 10. Final arterial blood gas, including lactate, will be drawn at completion of |
| | VO₂ max protocol (10 ± 1 minutes post-exercise at altitude). |
| | 11. Chamber will return to ground level atmospheric pressure. |
| | 12. Subjects will complete a post-exposure survey inquiring about side effects |
| | and perceived performance. |
| | 13. Arterial catheter and IV will be removed. |
| | Adverse event monitoring will begin from check in at the site on Treatment day |
| | 1 and through the follow up phone call 48 hr (±1 day). |
| Statistical | Subjects will be randomized, maintaining a balance of enrollment across each |
| Analysis Plan | TSC dosing cohort (n = 10 per dose cohort) as well as balance of altitude |
| | exposure sequence. Subjects who fail to complete both altitude exposure |
| | sessions could be replaced to ensure a per protocol analyses of n = 30 subjects. |
| | Primary analyses will be based on sequence matched changes: |
| | Median PaO₂ per cohort TSC vs PBO |
| | Median VO₂ per cohort TSC vs PBO |
| | Secondary analyses based on sequence matched changes: |
| | Median SpO₂ per cohort TSC vs PBO |
| | Median Lactate per cohort TSC vs PBO |
| | Observational |
| | PaO₂ per subject TSC vs PBO |
| | VO₂ per subject TSC vs PBO |

*Potential carry-over and sequence effect is acknowledged and mitigation plans include extended rest and washout period between dose and altitude exposures, and baseline variables re-established at each altitude exposure.

### Study Rationale

This study will evaluate the effectiveness of TSC in enhancing oxygen delivery to the blood and tissues during exercise under hypoxic conditions. Arterial partial pressure of oxygen in the blood (PaO₂), will reliably measure the uptake of oxygen dissolved into the blood from inspired gases. Studying TSC at altitude in exercising subjects will allow determination of the extent to which TSC can increase oxygenation at both uptake and delivery ends of the oxygenation path: at intake from the lungs to the blood, and from the blood to peripheral tissue.

This clinical trial is designed to determine the effect of TSC on PaO₂ and VO₂ max in healthy volunteers exercising at altitude.

### Study Objectives

### Primary Objectives

To determine the effect of TSC on PaO₂ and VO₂ max in healthy volunteers exercising at altitude.

### Secondary Objectives

To assess the effect of TSC on oxygen saturation (SpO₂) and lactate in healthy volunteers exercising at altitude.

### Study Endpoints

### Primary Endpoints

Difference in PaO₂ and VO₂ max between the control and experimental exposures.

### Secondary Endpoints

Differences in oxygen saturation (SpO₂) and lactate between the control and experimental exposures.

### Safety Endpoints

Adverse Events (AE), and Serious Adverse Events (SAE) will be reported per protocols. Laboratory values (complete blood count [CBC], basic metabolic panel [BMP] HIV, HBsAg, HCVAb), vital signs (blood pressure, heart rate, respiratory rate, temperature), and oxygen saturation (SpO₂) will be monitored.

### Study Design

The subject's maximum duration of participation is expected to be approximately 32 days (maximum 28 days for screening, 1 day of treatment, and maximum 3 days for follow up). The study completion date is defined as the date on which the last subject in the study completes the final protocol-defined assessment(s). This includes the follow-up visit or contact, whichever is later.

This is a double-blind, randomized, placebo-controlled crossover study designed to investigate TSC's effects on oxygen enhancement using an experimental model to induce hypoxia in study participants. The primary endpoints measured in this study are maximal oxygen consumption and partial pressure of arterial blood oxygen in normal healthy volunteers subjected to incremental levels of physical exertion while exposed to hypoxic and hypobaric conditions at a simulated altitude of 15,000 feet above sea level. The secondary endpoints are to assess the effect of TSC on SpO₂ and lactate.

A total of 30 healthy volunteers are enrolled in the trial with each subject serving as their own control by completing the experiment twice in a random, blinded order in the same day with a 3-hour rest and wash out period between experimental intervals. During one ascent, study subjects receive intravenous (IV) placebo administration and the other ascent the same subject receives a single IV dose of TSC at one of three dose levels (0.5 mg/kg, 1.5 mg/kg, or 2.5 mg/kg).

### Study Population

Subjects will be randomized to a single IV bolus dose of TSC (0.5 mg/kg, 1.5 mg/kg, 2.5 mg/kg). Each subject will complete the experiment twice in a random order, thereby serving as their own control. One time they will receive TSC and the other placebo normal saline.

### Study Drug

TSC is dosed based on the subject's baseline weight, obtained on the day of screening, on a milligram per kilogram basis.

### Randomization

Volunteers will be randomized to one of 3 TSC doses with placebo crossover in a 1:1:1 schema.

This is a prospective, randomized control, crossover trial. Each subject will complete the experiment twice in a random order, thereby serving as their own control. Randomization of study drug administration as well as exposure sequence schedule on treatment Day 1 will be assigned by study statistician. Investigators and subjects will be blind to the randomization schedule unless circumstances arise affecting the health and safety of subjects.

### Dosing Regimen

Study drug will be administered as a one-time IV bolus injection to 10 unique subjects per dose cohort, to include 3 TSC dose levels. As this is a crossover trial, each subject will complete the experiment twice, in random order, thereby serving as their own control. Placebo will consist of 7 mL a volume matched dose of normal saline per TSC dosing cohort. One time they will receive TSC and the other placebo normal saline. Subjects will be randomized to the following dose cohorts:

| | # of Subjects in | TSC Dose | |
|---|---|---|---|
| Dosing Cohort | Cohort | (mg/kg) | Placebo |
| 1 | 10 | 0.5 | Volume matched |
| | | | dose |
| 2 | 10 | 1.5 | Volume matched |
| | | | dose |
| 3 | 10 | 2.5 | Volume matched |
| | | | dose |

### Laboratory

Clinical laboratory evaluations:
Fasting is not required before collection of laboratory samples
Blood samples will be collected at the time points indicated in the protocol per institutional process and procedures.

### Primary and Secondary Efficacy Analyses

Primary analyses will be based on sequence matched changes in median PaO₂ and median VO₂ following a single administration of TSC versus placebo in subjects exposed to simulated altitude and undergoing exercise. This is a crossover design, repeated measures study where each subject will serve as their own control. Each subject will be randomized to a TSC dose and will also be randomized to their altitude exposure sequence on the day of testing.

Secondary Analyses based on sequence matched changes:
Median SpO₂ per cohort TSC vs PBO
Median Lactate per cohort TSC vs PBO

Observational:
PaO₂ per subject TSC vs PBO
VO₂ per subject TSC vs PBO
   *Potential carry-over and sequence effect is acknowledged and mitigation plans include extended rest and washout period between exposures, and baseline variables re-established for each exposure.

### Results

Fifteen of 30 planned subjects (11 male, 4 female, mean±SD age 24.6±5.6) have completed the experimental protocol. Time from baseline VO₂ₘₐₓ to altitude exposure is 13.0±9.1 days. Mean baseline VO₂ₘₐₓ is 46.0±9.3 ml/kg/min vs. 30.5±5.4 ml/kg/min at altitude (33.3%±4.6% decrement). Peak HR is 184±7 at baseline vs. 174±9 at altitude (5.2%±3.7% decrement). Peak RER is 1.26±0.06 at baseline vs. 1.47±0.18 at altitude (17.4%±15.0% increase at altitude). PaO₂ is 111±7 at surface vs 48±4 prior to start of exercise at altitude and 46±4 at completion of exercise.

Results with 30 Subjects

Following exercise under hypoxic conditions, an increase in pH and a decrease in lactate are observed in the study subjects treated with the highest dose of TSC (2.5 mg/kg), both at the end of the exercise period and at 10 minutes post-exercise. These data suggest the 2.5 mg/kg dose of TSC decreases blood acidity (i.e., lactic acid accumulation) and enhances metabolic recovery at 10 minutes after completion of exercise under the stressful conditions of simulated high altitude and exercise.

These positive changes observed in blood markers of oxygen utilization results suggest TSC may enhance oxygen availability at the cellular level.

Specific results include the following:

Positive effects on lactate and pH are observed with the TSC 2.5 mg/kg dose at the end of the exercise period versus the baseline measurement. The effects on pH are significantly different (p<0.1) when compared to placebo.
A “carry-over” effect is observed in subjects who received TSC in the first treatment (“ascent”) of the day versus those who received placebo first and TSC for the second ascent. The change in pH from baseline to last study measurement is also significantly lower (p<0.05) when placebo is administered for the first ascent of the day.
The 2.5 mg/kg dose appears to have a positive effect on post-exercise recovery based on comparison of the measurements for pH, lactate, oxygen saturation (SpO₂) and other markers at 10 minutes post-exercise versus last exercise measurements.
There are no observed changes among subject's intra-day maximal oxygen consumption tests or in subject's partial pressure of arterial blood oxygen.
TSC is safe and well tolerated at all doses tested in the study with no serious adverse events reported.

Further results include the following:
Carry-over effect of active:
   A sequence effect is detected with pH based on the change from baseline. Subjects who receive placebo first have a significantly greater reduction from baseline compared to subject who receive placebo second (p<0.05).
   A sequence effect is detected with HCO3 based on the change from baseline. Subjects who receive placebo first had a greater reduction from baseline compared to subjects who receive placebo second that is highly suggestive (p<0.1).
   While not significant nor suggestive statistically, subjects who receive placebo first have a greater reduction from baseline compared to subjects who receive placebo second for oxygen saturation and PACO2.
Difference in last exercise value between active and placebo doses:
   For PAO2, the 2.5 mg/kg TSC dose has the lowest least square mean value compared to the other TSC doses and placebo. The 2.5 mg/kg TSC dose separates statistically from the 1.5 mg/kg TSC dose.
   For oxygen saturation, the 2.5 mg/kg TSC dose has the lowest least square mean value compared to the other TSC doses and placebo. The 2.5 mg/kg TSC dose separates from the 1.5 mg/kg TSC dose with a probability value that is highly suggestive of a significant difference (p<0.1).
   For lactic acid, there is a trend of lower lactic acid with an increasing dose of TSC. The 0.5 mg/kg TSC dose results are very similar to the placebo dose. The 2.5 mg/kg TSC dose has the lowest least square mean value compared to the other TSC doses and placebo. The 2.5 mg/kg TSC dose separates from the 0.5 mg/kg TSC dose with a probability value that is highly suggestive of a significant difference (p<0.1).
   For PACO2, the 2.5 mg/kg TSC dose has the highest least square mean value compared to the other TSC doses and placebo.
   For pH, the 2.5 mg/kg TSC dose has the highest least square mean value compared to the other TSC doses and placebo.
   For HCO3, the 2.5 mg/kg TSC dose has the highest least square mean value compared to the other TSC doses and placebo. The 2.5 mg/kg TSC dose separates statistically from the 0.5 mg/kg TSC dose.
Difference in last exercise value minus baseline between active and placebo doses:
   For PAO2, the 0.5 mg/kg TSC dose and placebo have the lowest least square mean value compared to the other TSC doses.
   For VO2 maximum, the 2.5 mg/kg TSC dose have the lowest least square mean value compared to the other TSC doses and placebo.
   For oxygen saturation, the 2.5 mg/kg TSC dose has the lowest least square mean value compared to the other TSC doses and placebo.
   For lactic acid, there is a trend of lower lactic acid with an increasing dose of TSC. The 2.5 mg/kg TSC dose has the lowest least square mean value compared to the other TSC doses and placebo.
   For pH, the 2.5 mg/kg TSC dose has the highest least square mean value compared to the other TSC doses and placebo. The 2.5 mg/kg TSC dose separates statistically from the 0.5 mg/kg TSC dose.
   For HCO3, the 2.5 mg/kg TSC dose has the highest least square mean value compared to the other TSC doses and placebo.
Difference in post-exercise value between active and placebo doses:
   For oxygen saturation, the 2.5 mg/kg TSC dose has the highest least square mean value compared to the other TSC doses and placebo.
   For lactic acid, there is a trend of lower lactic acid with an increasing dose of TSC. The 2.5 mg/kg TSC dose has the lowest least square mean value compared to the other TSC doses and placebo. The 2.5 mg/kg TSC dose separates statistically from the 0.5 mg/kg TSC dose (p<0.05). The 2.5 mg/kg TSC dose separates from the placebo with a probability value that is highly suggestive (p=0.01).
   For PACO2, the 2.5 mg/kg TSC dose has the highest least square mean value compared to the other TSC doses and placebo. The 2.5 mg/kg TSC dose separates statistically from the 0.5 mg/kg TSC dose (p<0.05).
   For pH, the 2.5 mg/kg TSC dose has the highest least square mean value compared to the other TSC doses and placebo. The 2.5 mg/kg TSC dose separates statistically from the 0.5 mg/kg TSC dose. The 2.5 mg/kg TSC dose separates from placebo with a probability value that is highly suggestive (p=0.01).
   For HCO3, there is a trend of higher values with an increasing dose of TSC. The 2.5 mg/kg TSC dose has the highest least square mean value compared to the other TSC doses and placebo. The 2.5 mg/kg TSC dose separates statistically from the 0.5 mg/kg TSC dose.
Difference in 10-minute post-exercise change from baseline between active and placebo doses:
   For PAO2, there is a trend of higher values with an increasing dose of TSC. The 2.5 mg/kg TSC dose has the highest least square mean value compared to the other TSC doses and placebo. The 2.5 mg/kg TSC dose separates statistically from the 0.5 mg/kg TSC dose.
   For oxygen saturation, there is a trend of greater oxygen saturation with an increasing dose of TSC. The 2.5 mg/kg TSC dose has the highest least square mean value compared to the other TSC doses and placebo; the change from baseline is negative for all doses and placebo except the 2.5 mg/kg TSC dose. The 2.5 mg/kg TSC dose separates statistically from the 0.5 mg/kg TSC dose (p<0.05). The 2.5 mg/kg TSC dose separates from placebo with a probability value that is highly suggestive (p=0.01).
   For lactic acid, there is a trend of lower lactic acid with an increasing dose of TSC. The 2.5 mg/kg TSC dose has the lowest least square mean value compared to the other TSC doses and placebo. The 2.5 mg/kg TSC dose separates statistically from the 0.5 mg/kg TSC dose (p<0.05).
   For pH, the 2.5 mg/kg TSC dose has the highest least square mean value (smallest reduction from baseline) compared to the other TSC doses and placebo. The 2.5 mg/kg TSC dose separates statistically from the 0.5 mg/kg TSC dose. The 2.5 mg/kg TSC dose separates from placebo with a probability value that is highly suggestive (p=0.01).
   For HCO3, there is a trend of higher values (smallest reduction from baseline) with an increasing dose of TSC. The 2.5 mg/kg TSC dose has the highest least square mean value compared to the other TSC doses and placebo.
Difference based on the change: last exercise to 10-minutes post-exercise:
   For PAO2, the 2.5 mg/kg TSC dose has the highest least square mean value compared to the other TSC doses and placebo.
   For oxygen saturation, there is a trend of greater oxygen saturation with an increasing dose of TSC. The 2.5 mg/kg TSC dose has the highest least square mean value compared to the other TSC doses and placebo.
   For lactic acid, the 2.5 mg/kg TSC dose and the 1.5 mg/kg TSC dose have the lowest least square mean value compared to the other TSC doses and placebo.
   For PACO2, the 2.5 mg/kg TSC dose has the highest least square mean value compared to the other TSC doses and placebo.
   For pH, the 2.5 mg/kg TSC dose has the smallest change from the last recorded value while exercising and the 10-minute post exercise value.
   For HCO3, there is a trend of higher values with an increasing dose of TSC. The 2.5 mg/kg TSC dose has the highest least square mean value compared to the other TSC doses and placebo.

### Example 3

### Protocol Synopsis

| | |
|---|---|
| Title of Study | Double-blind, Placebo-Controlled Study of Trans Sodium Crocetinate in |
| | Patients with Interstitial Lung Disease |
| Study | The objective of the study is to determine the effect of trans sodium |
| Objective | crocetinate (TSC) on lung function as measured by diffusing capacity of the |
| | lungs for carbon monoxide (DLCO), the 6-minute walk test (6MWT), and |
| | heart rate recovery (HRR) in patients with interstitial lung disease (ILD) |
| Endpoints | Primary |
| | Change from baseline at 30 minutes in DLCO after administration of |
| | a single dose of TSC in patients with ILD |
| | Secondary |
| | Change from baseline in 6MWT after administration of a single dose |
| | of TSC in patients with ILD |
| | Change from baseline in the Borg Scale after the 6MWT |
| | Change from baseline in HRR after each 6MWT |
| | Tertiary |
| | Change from baseline at 10 minutes in DLCO after administration of |
| | a single dose of TSC in patients with ILD |
| Study | Patients between the ages of 30 and 85 years with ILD (previously |
| Population | diagnosed) |
| Participant | 32 days (inclusive of screening visit, office dosing/DLCO visit, and follow- |
| Duration | up phone call) |
| Eligibility | Inclusion Criteria |
| | 1. Male or Female, age 30-85 years at screening |
| | 2. Able to provide informed consent and agree to adhere to all study visits |
| | and requirements |
| | 3. Females of childbearing potential must have a negative blood pregnancy |
| | test at screening and agree to use one of the accepted contraceptive |
| | regimens, or a double method of birth control during the study and at |
| | least 30 days after the last dose of study drug |
| | 4. Established diagnosis of ILD (clinical, radiographic, or histologic) |
| | 5. SpO2 ≥88% at rest by pulse oximetry while breathing ambient air |
| | 6. Free of any active cardiovascular or neuromuscular disease, at PI |
| | discretion |
| | 7. Clinically stable disease with no major medication changes in the last |
| | 4 weeks |
| | 8. Forced vital capacity (FVC) ≥45% of predicted (within past 6 months) |
| | 9. DLCO corrected for hemoglobin 30-90% of predicted, inclusive (within |
| | past 6 months) |
| | 10. Sars-CoV-2 negative at screening |
| | Exclusion Criteria |
| | 1. Known allergy to study medication |
| | 2. Pregnancy or lactation |
| | 3. Current smoker |
| | 4. Inability to perform pulmonary function testing |
| | 5. Active infection at screening or day of study visit |
| | 6. Known pulmonary hypertension (PH) requiring PH-specific treatment |
| | 7. AST/ALT ≥3x ULN and/or total bilirubin ≥2x ULN |
| | 8. Received any investigational medicine (IMP) within past 30 days |
| | 9. Surgery or hospitalization in past 3 months determined by the PI to be |
| | clinically relevant. |
| | 10. Current alcohol or substance abuse |
| | 11. Known active or latent hepatitis B or C |
| | 12. History of end-stage liver or renal disease |
| | 13. Positive COVID test anytime within 3 months of screening. Note: |
| | Patients who were previously vaccinated for COVID are allowed |
| | 14. History of venous thromboembolic disease |
| | 15. History of acute or chronic ophthalmologic conditions currently |
| | requiring treatment |
| Safety | Assessment of adverse events beginning at check in on the day of TSC |
| | dosing until the patient completes the study or withdraws prematurely. |
| Study Design | Randomized, double-blind, placebo-controlled, study comparing the |
| | response of patients randomized to receive 2.5 mg/kg of TSC to patients |
| | randomized to receive 7 mL of normal saline (placebo). |
| Study | A maximum of 27 patients with ILD will be recruited to participate in |
| Overview | this study. Following written informed consent and screening, baseline |
| | assessments will be obtained, including DLCO, forced vital capacity |
| | (FVC), SpO2, physical exam, ophthalmologic assessment, laboratory |
| | (complete blood count [CBC] and comprehensive metabolic panel |
| | [CMP]), urine drug screen (UDS), serum pregnancy test for females of |
| | childbearing potential, and 12-lead electrocardiogram (ECG). |
| | On the day of testing, an indwelling IV catheter will be placed for study |
| | drug administration. Prior to the administration of the study drug (TSC or |
| | placebo) a 12-lead ECG will be repeated and vital signs (blood pressure |
| | [BP], heart rate [HR], respiratory rate [RR] and temperature [Temp]) will |
| | be recorded. |
| | 60 minutes prior to study drug dosing, a 6MWT will be administered, |
| | with SpO2 monitoring, Borg scale for dyspnea, and HRR, will be |
| | recorded. |
| | 30 minutes prior to study drug dosing, DLCO will be measured. |
| | After baseline testing has been completed, the study drug (TSC or |
| | placebo) will be administered. The study drug will be administered as |
| | a single IV bolus injection to patients. |
| | Repeat DLCO measurements will be recorded at 10 min and 30 min |
| | after administration of the study drug. The 6MWT will be performed |
| | at 60 min after administration of the study drug. Pulse oximetry will |
| | be recorded from baseline and throughout the DLCO evaluation and |
| | before and after each 6MWTs. Blood pressure and heart rate will be |
| | measured at baseline (pre-testing), pre- and post- each DLCO |
| | measurement and 6MWT (with HRR measured after each 6MWT as |
| | well). Adverse events will be assessed. |
| | Each DLCO measurement will be comprised of 2 DLCO readings; a |
| | valid measurement requires ≤10% difference between the readings |
| | (Rochwerg, B. et al., Eur Respir J, 2017, 50, 1602426) |
| | Each 6MWT will be performed in compliance with local practice |
| | standards at site. |
| | Safety assessment at 48 h (+2 d) to be obtained with phone call. |
| Statistical | Primary Objective: Compare the proportion of patients who achieve a pre- |
| | specified level of improvement in DLCO 30 minutes after administration of |
| | the study drug (TSC or placebo) between the 2 randomized treatment arms. |
| Analysis Plan | Secondary Objective 1: Compare the proportion of patients who achieve a |
| | pre-specified level of improvement in 6MWD after administration of the |
| | study drug (TSC or placebo) between the 2 randomized treatment arms. |
| | Secondary Objective 2: Compare the proportion of patients who achieve a |
| | pre-specified level of improvement in the Borg dyspnea scale after |
| | administration of the study drug (TSC or placebo) between the 2 randomized |
| | treatment arms. |
| | Secondary Objective 3: Compare the proportion of patients who achieve a |
| | pre-specified level of improvement in HRR after administration of the study |
| | drug (TSC or placebo) between the 2 randomized treatment arms. |
| | Secondary Examination of Objective 1: Compare the median change from |
| | baseline in the 6MWT between the 2 randomized treatment arms. |
| | Secondary Examination of Objective 2: Compare the median change from |
| | baseline after the 6MWT in the Borg Scale score between the 2 randomized |
| | treatment arms. |
| | Secondary Examination of Objective 3: Compare the median change from |
| | baseline after the 6MWT in HRR between the 2 randomized treatment arms. |

### Introduction

This study will evaluate the effectiveness of TSC in enhancing oxygen delivery to the blood and tissues to patients with ILD. Measuring DLCO is a well-characterized method of assessing the ability of the lungs to transfer gas from inhaled air to the red blood cells in pulmonary capillaries. The 6MWT is a standard test that evaluates the pulmonary system during exercise.

### Study Rationale

This clinical trial is designed to determine the effect of TSC on DLCO and 6 MWT from baseline after administration of a single dose of TSC in ILD patients. Study assessments include DLCO, 6MWT, Borg dyspnea scale, and heart rate recovery (HRR). This study will evaluate the effectiveness of TSC in enhancing oxygen delivery to the blood and tissues in patients with confirmed ILD. Measuring the diffusion rate of CO is a well-characterized method of assessing the ability of the lungs to transfer gas from inhaled air to the red blood cells in pulmonary capillaries. The 6MWT is a standard test to measure the distance that a patient can quickly walk on a flat, hard surface in a period of 6 minutes. It evaluates the global and integrated responses of all the systems involved during exercise, including the pulmonary system. The Borg scale is a measure of perceived exertion. HRR is a measure of how quickly the heart rate returns to baseline after exertion. Each of these measures are expected to be impacted in patients with ILD.

### Objectives and Endpoints

### Study Objectives

The objective of the study is to determine the effect of trans sodium crocetinate (TSC) on lung function as measured by diffusing capacity of the lungs for carbon monoxide (DLCO), the 6-minute walk test (6MWT), and heart rate recovery (HRR) in patients with interstitial lung disease (ILD).

### Study Endpoints

### Primary Endpoint

Change from baseline at 30 minutes in DLCO after administration of a single dose of TSC in patients with ILD

### Secondary Endpoints

1. Change from baseline in 6MWT after administration of a single dose of TSC in patients with ILD
2. Change from baseline in the Borg Scale after the 6MWT
3. Change from baseline in HRR after each 6MWT

### Tertiary Endpoint

1. Change from baseline at 10 minutes in DLCO after administration of a single dose of TSC in patients with ILD

### Safety Endpoint

1. Assessment of adverse events beginning at check-in on the day of TSC dosing until the patient completes the study or withdraws prematurely.

### Study Design

This study is a randomized, double-blind, placebo-controlled, single dose study. Following Informed Consent procedures, patients will be screened for eligibility. Eligible patients will undergo baseline assessments, be randomized 2:1 TSC:placebo, receive a single infusion of study drug, and undergo safety and efficacy assessments. They will be followed up by phone 48 hours later.

### Study Drug

### Allocation of Patients to Treatment

Patients will be randomized to TSC (2.5 mg/kg) or placebo in a 2:1 schema.

### Dosing Regimen

Study drug will be administered as a one-time IV bolus injection.

### Study Assessments and Procedures

### Treatment Visit (Day 1)

### Clinic Arrival

Inclusion/Exclusion criteria
Concomitant medications
Vital signs (BP, HR, RR, Temp)
Urine pregnancy test for WOCBP
COVID-19 test
12-lead ECG
Place intravenous catheter
Randomization

### Baseline

### Sixty (60) Minutes Prior to Study Drug Administration

Concomitant medications
Vital signs (BP, RR, HR)
6MWT with SpO₂
Borg scale
HRR

### Thirty (30) Minutes Prior to Study Drug Administration

Concomitant medications
Vital signs (BP, HR, RR)
DLCO

### Treatment

### Allocation of Patients to Treatment

Once the baseline evaluations are complete, study drug will be administered as a single IV bolus injection to patients.

Blood samples for PK will be collected.

Repeat DLCO will be measured at 10 min and 30 min after study drug administration. Repeat 6MWT will be performed at 60 min after study drug administration. Continuous pulse oximetry will be recorded. Blood pressure and heart rate will be measured at baseline (pre-testing), and then pre- and post- each DLCO and 6MWT (with HRR measured after each 6MWT as well). Adverse events will be assessed starting at study day check-in and through a post-study visit 48 hr follow up phone call.

### Efficacy

DLCO: One of the most clinically valuable tests of lung function. The DLCO measures the ability of the lungs to transfer gas from inhaled air to the red blood cells in pulmonary capillaries. A trace amount of CO, which is taken up in the alveoli, is mixed with an inert gas which does not diffuse into the lung, and is inhaled by the patient. The rate of diffusion is measured by analyzing the concentrations of carbon monoxide and inert gas in the inspired gas and in the exhaled gas and takes into account the volume of the alveoli (Barratt, S. et al., Respir Med, 2018, 135, 51-56; Saydain, G. et al., Chest, 2004, 125 (2), 446-452).

6MWT: The 6MWT is a simple test that requires no exercise equipment or advanced training for technicians. This test measures the distance that a patient can quickly walk on a flat, hard surface in a period of 6 minutes (the 6MWD). It evaluates the global and integrated responses of all the systems involved during exercise, including the pulmonary system. The self-paced 6MWT assesses the submaximal level of functional capacity. Most patients do not achieve maximal exercise capacity during the 6MWT; instead, they choose their own intensity of exercise and are allowed to stop and rest during the test (Am Respir Crit Care Med, 2002, 166 (1), 111-117 and Erratum in Am J Respir Crit Care Med, 2016, 193 (1), 1185). Each test encompasses two single-breath tests that must be within 10% of each other for validation (Rochwerg, B. et al., Eur Respir J, 2017, 50, 1602425).

Borg Scale: This is a patient-reported measure of dyspnea. It is a 10 point scale of perceived shortness of breath that ranges from none (as would be a level of dyspnea expected to sit and read a book) to very, very short of breath (like the amount of dyspnea in the final kick to finish a race and could not be maintained for a long period of time).

HRR: HRR is the decrease in heart rate as it returns to baseline following strenuous activity. Heart rate will be recorded before and after the 6MWT and the change from baseline recorded.

### Pharmacokinetics

### PK Timepoints

All timepoints are post-administration of study drug
5 min (+1-2 min window, but not before 5 min post-administration of study drug)
15 min (±3 min)
45 min (±3 min)
75 min (±3 min)

### PK Procedures

Blood samples of approximately 6 mL will be collected at each timepoint for measurement of plasma concentrations of TSC.

The actual date and time will be recorded by 24-hour clock time noting the hour and minute of the end of the TSC infusion.

The same digital clock will be consistently used to record the actual hour and minute of the start of each blood specimen collection.

Each sodium heparinized vacutainer blood collection tube will be clearly labeled with the patient identifier number, date and time point of the specimen collection.
1. About 6 mL samples of blood will be collected in a sodium heparinized vacutainer tube and the blood will be centrifuged to separate plasma.
2. The plasma will be separated into two approximately equal volumes in separate tubes. The plasma should be approximately 1.5 mL in each tube.
3. Protocol-specific instructions will be provided to the clinical site in a lab manual.
4. The plasma samples will be frozen at −70° C. One aliquot will be shipped to the bioanalytical lab identified in the PK Laboratory Manual, and the other stored at −70° C. as a back-up.
5. PK samples that are remaining after all PK analyses have been completed, may be used for additional analysis. Participant confidentiality will be maintained.

### Clinical Laboratory Tests

All clinical laboratory tests will be performed according to the laboratory's standard procedures. Reference ranges will be supplied by the laboratory and used to assess the results for clinical significance and out-of-range changes which may be associated with, or constitute, an AE. The investigator should assess out-of-range clinical laboratory values for clinical significance, indicating if the value(s) is/are not clinically significant or clinically significant. Abnormal clinical laboratory values, which are unexpected or not explained by the patient's clinical condition, may, at the discretion of the investigator or sponsor, be repeated as soon as possible until confirmed, explained, or resolved.
Clinical laboratory evaluations:
   Fasting is not required before collection of laboratory samples
   Blood will be collected at the time points indicated in the protocol
Venipuncture volumes:

| Day +/− Window | Laboratory Evaluation | Total Volume |
|---|---|---|
| Screening | Complete blood count (CBC), | 9 mL |
| (Day −28 to Day 0) | liver function tests (LFTs), basic | |
| | metabolic panel (BMP) | |
| Total all study days | CBC, BMP + LFTs | 9 mL |

It will be readily apparent to those skilled in the art that numerous modifications and additions can be made to both the present compounds and compositions, and the related methods without departing from the invention disclosed.

What is claimed is:

1. A method of improving peripheral tissue oxygenation in a human in need thereof, wherein the method comprises administering 2.5-5 mg/kg of trans sodium crocetinate to the human.

2. A method of treating interstitial lung disease in a human in need thereof, wherein the method comprises administering a therapeutically effective amount of a diffusion enhancing compound to the human.

3. A method of increasing distance covered in a 6-minute walk test in a human in need thereof, wherein the method comprises administering a therapeutically effective amount of a diffusion enhancing compound to the human.

4. A method of treating dyspnea in a human in need thereof, wherein the method comprises administering a therapeutically effective amount of a diffusion enhancing compound to the human.

5. A method of improving oxygen transfer efficiency from the aveoli of the lungs to hemoglobin within red blood cells in a human in need thereof, wherein the method comprises administering 2.5-5 mg/kg of trans sodium crocetinate to the human.

6. A method of increasing oxygen transfer from the lungs to the blood in a human in need thereof, wherein the method comprises administering 2.5-5 mg/kg of trans sodium crocetinate to the human.

7. A method of increasing oxygen transfer from blood to peripheral tissue in a human in need thereof, wherein the method comprises administering 2.5-5 mg/kg of trans sodium crocetinate to the human.

8. A method of improving heart rate recovery in a human in need thereof, wherein the method comprises administering a therapeutically effective amount of a diffusion enhancing compound to the human.

9. A method of treating, preventing, or reducing the amount of ischemia resulting from surgery in a human in need thereof, wherein the method comprises administering to the human 2.5-5 mg/kg of trans sodium crocetinate before, during, or after surgery a therapeutically effective amount of a diffusion enhancing compound.

10. A method of treating cancer (including brain and pancreatic cancer) in a human in need thereof, wherein the method comprises administering 2.5-5 mg/kg of trans sodium crocetinate to the human with chemotherapy and/or radiotherapy.
